(19) United States
(12) Patent Application Publication
(10) Pub. No.: US 2005/0147985 A1
(43) Pub. Date: Jul. 7, 2005

(54) Mixtures of isobarically labeled analytes and fragments ions derived therefrom

(75) Inventors: Darryl J. C. Pappin, Boxborough, MA (US); Subhasish Purkayastha, Acton, MA (US); James M. Coull, Westford, MA (US)

Correspondence Address: APPLIED BIOSYSTEMS, 500 OLD CONNECTICUT PATH, FRAMINGHAM, MA, 01701, US

(73) Assignee: Applera Corporation

(21) Appl. No.: 10/822,639

(22) Filed: Apr. 12, 2004

Related U.S. Application Data
(63) Continuation-in-part of application No. 10/751,353, filed on Jan. 5, 2004.

(51) Int. Cl. C12Q 1/68; C07H 21/02; G01N 33/00; C07J 43/00

(52) U.S. Cl. 435/6; 436/86; 530/409; 536/23.1; 540/107; 544/359

(57) ABSTRACT

This invention pertains to mixtures of isobarically labeled analytes and fragment ions thereof.

### CROSS REFERENCE TO RELATED APPLICATIONS

This application is a continuation-in-part of U.S. patent application Ser. No. 10/751,353, filed on Jan. 5, 2004, incorporated herein by reference.

### FIELD OF THE INVENTION

This invention pertains to mixtures of isobarically labeled analytes and fragment ions thereof.

### INTRODUCTION

Isotopically enriched N-substituted piperazines and N-substituted piperazine acetic acids can be used as intermediates in the synthesis of active esters of N-substituted piperazine acetic acid. Active esters are well known in peptide synthesis and refer to certain esters that are easily reacted with an amine of an amino acid under conditions commonly used in peptide synthesis (For a discussion of active esters please see: Innovation And Perspectives In Solid Phase Synthesis, Editor: Roger Epton, SPCC (UK) Ltd, Birmingham, 1990). A commonly used form of an active ester is the N-hydroxysuccinimidyl (NHS) ester.

The active esters of N-substituted piperazine acetic acid can be used as labeling reagents. In some embodiments, a set of isobaric labeling reagents can be prepared. The set of isobaric labeling reagents can be used to label analytes, such as peptides, proteins, amino acids, oligonucleotides, DNA, RNA, lipids, carbohydrates, steroids, small molecules and the like. The labeled analytes can be mixed together and analyzed simultaneously in a mass spectrometer. In some embodiments, this invention pertains to mixtures of isobarically labeled analytes and fragment ions thereof.

Because the heavy atom isotope distribution in each of the isobaric labeling reagents can be designed to result in the generation of a unique “signature ion” when analyzed in a mass spectrometer (MS), labeled components of the mixture associated with each of the labeling reagents, and by implication components of each labeling reaction used to produce the mixture, can be deconvoluted. Deconvolution can include determining the relative and/or absolute amount (often expressed in concentration or quantity) of one or more labeled components in each of the individual samples that were labeled and combined to form the mixture. The active esters of N-substituted piperazine acetic acid described herein therefore can be powerful tools for analyte analysis. Experimental analysis for which these reagents can be used includes, but is not limited to, multiplex proteomic analysis, mudpit experiments, affinity pull-downs, determination of post-translational modifications (PTMs) and multiple control experiments.

### BRIEF DESCRIPTION OF THE DRAWINGS

FIG. 1 is an illustration of a synthetic scheme for the synthesis of N-methyl piperazines.

FIG. 2A is an illustration of a synthetic scheme for the synthesis of N-methyl piperazine acetic acids.

FIG. 2B is an illustration of another synthetic scheme for the synthesis of N-methyl piperazine acetic acids.

FIG. 2C is an illustration of yet another synthetic scheme for the synthesis of N-methyl piperazine acetic acids.

FIG. 3A is an illustration of a synthetic scheme for the synthesis of ¹⁸O labeled N-methyl piperazine acetic acids.

FIG. 3B is an illustration of another synthetic scheme for the synthesis of ¹⁸O labeled N-methyl piperazine acetic acids.

FIG. 4A is an illustration of a synthetic scheme for the synthesis of various active esters of N-methyl piperazine acetic acid.

FIG. 4B is an illustration of another synthetic scheme for the synthesis of various active esters of N-methyl piperazine acetic acid.

FIG. 4C is an illustration of yet another synthetic scheme for the synthesis of various active esters of N-methyl piperazine acetic acid.

FIG. 4D is an illustration of still another synthetic scheme for the synthesis of various active esters of N-methyl piperazine acetic acid.

FIG. 5A is an illustration of the heavy atom isotope incorporation pathway for the preparation of four isobaric N-methyl piperazine acetic acids.

FIG. 5B is an illustration of the labeling and fragmentation of peptides using four isobaric N-methyl piperazine acetic acid active ester labeling reagents.

FIG. 5C is an illustration of two possible structures for each of the signature ions produced from the isobaric set of reagents illustrated in FIG. 5B.

### DEFINITIONS

For the purposes of interpreting of this specification, the following definitions will apply and whenever appropriate, terms used in the singular will also include the plural and vice versa:

As used herein, “analyte” refers to a molecule of interest that may be determined. Non-limiting examples of analytes include, but are not limited to, proteins, peptides, nucleic acids (both DNA or RNA), carbohydrates, lipids, steroids and other small molecules with a molecular weight of less than 1500 Daltons (Da). The source of the analyte, or the sample comprising the analyte, is not a limitation as it can come from any source. The analyte or analytes can be natural or synthetic. Non-limiting examples of sources for the analyte, or the sample comprising the analyte, include cells or tissues, or cultures (or subcultures) thereof. Non-limiting examples of analyte sources include, but are not limited to, crude or processed cell lysates, body fluids, tissue extracts, cell extracts or fractions (or portions) from a separations process such as a chromatographic separation, a 1D electrophoretic separation, a 2D electrophoretic separation or a capillary electrophoretic separation. Body fluids include, but are not limited to, blood, urine, feces, spinal fluid, cerebral fluid, amniotic fluid, lymph fluid or a fluid from a glandular secretion. By processed cell lysate we mean that the cell lysate is treated, in addition to the treatments needed to lyse the cell, to thereby perform additional processing of the collected material. For example, the sample can be a cell lysate comprising one or more analytes that are peptides formed by treatment of the cell lysate with a proteolytic enzyme to thereby digest precursor peptides and/or proteins.

Except as when clearly not intended based upon the context in which it is being used (e.g. when made in reference to a structure that dictates otherwise), “ester” refers to both an ester and/or a thioester.

As used herein, “fragmentation” refers to the breaking of a covalent bond.

As used herein, “fragment” refers to a product of fragmentation (noun) or the operation of causing fragmentation (verb).

As used herein, “isotopically enriched” means that a compound (e.g. labeling reagent) has been enriched synthetically with one or more heavy atom isotopes (e.g. stable isotopes such as Deuterium, ¹³C, ¹⁵N, ¹⁸O, ³⁷Cl or ⁸¹Br). Because isotopic enrichment is not 100% effective, there can be impurities of the compound that are of lesser states of enrichment and these will have a lower mass. Likewise, because of over-enrichment (undesired enrichment) and because of natural isotopic abundance, there can be impurities of greater mass.

As used herein, “labeling reagent” refers to a moiety suitable to mark an analyte for determination. The term label is synonymous with the terms tag and mark and other equivalent terms and phrases. For example, a labeled analyte can be referred to as a tagged analyte or a marked analyte.

As used herein, “natural isotopic abundance” refers to the level (or distribution) of one or more isotopes found in a compound based upon the natural prevalence of an isotope or isotopes in nature. For example, a natural compound obtained from living plant matter will typically contain about 0.6% ¹³C.

As used herein, isobars are structurally and chemically indistinguishable compounds (except for isotopic content and/or distribution) of the same nominal gross mass. By comparison, isomers are structurally and/or chemically distinguishable compounds of the same nominal gross mass.

### DESCRIPTION OF VARIOUS EMBODIMENTS OF THE INVENTION

I. Preparation of N-Substituted Piperazines Comprising Heavy Atom Isotopes

In some embodiments, this invention pertains to a method for the production of isotopically enriched N-substituted piperazines, and the N-substituted piperazines themselves. According to the method, a partially protected amino acid can be condensed with an N-substituted amino acid ester wherein at least one of the two amino acids comprises a heavy atom isotope such as, for example, ¹⁸O, ¹⁵N, ¹³C, ⁸¹Br, ³⁷Cl or deuterium. When condensing the two amino acids, any side chain reactive groups can be protected as they would be for the condensation of amino acids to form peptides. Similarly, the condensation chemistry can be chosen from the various methods known for condensing amino acids. These include, but are not limited to, the use of carbodiimides (e.g. dicyclohexylcarbodiimide, DCC), active esters, mixed anhydride formation and the like.

The partially protected amino acid comprises an amine-protecting group (N-protecting group), such as tert-butyloxycarbonyl (t-boc); a well-known protecting group in peptide synthesis. Numerous other suitable N-protecting groups are well known in the peptide synthesis art. The partially protect amino acid can comprise a side chain protecting where the amino acid comprises a reactive side chain moiety. Numerous suitable side chain protecting groups are likewise known in the peptide synthesis art. The amino acid can be any natural amino acid (e.g. glycine, alanine, lysine) or non-natural amino acid of basic structure: 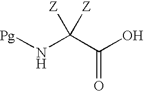
wherein Pg can be the N-protecting group. Each group Z can be independently hydrogen, deuterium, fluorine, chlorine, bromine, iodine, an amino acid side chain, a straight chain or branched C1-C6 alkyl group that may optionally contain a substituted or unsubstituted aryl group wherein the carbon atoms of the alkyl and aryl groups each independently comprise linked hydrogen, deuterium or fluorine atoms or a straight chain or branched C1-C6 alkoxy group that may optionally contain a substituted or unsubstituted aryl group wherein the carbon atoms of the alkyl and aryl groups each independently comprise linked hydrogen, deuterium or fluorine atoms. In some embodiments, each Z is independently hydrogen, methyl or methoxy. In some embodiments, each Z is hydrogen, deuterium, fluorine, chlorine, bromine or iodine. An alkyl ether group, as used herein, can include one or more polyethylene glycol substituents. Similarly, the alkoxy group, as used herein, can comprise ether and/or polyethylene glycol substituents. The N-protecting group can be an acid labile protecting group. The N-protecting group can be a base labile protecting group.

The N-substituted amino acid ester can be any natural amino acid (e.g. glycine, alanine, lysine) or non-natural amino acid of basic structure: 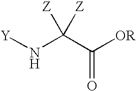
wherein Z is previously defined. The group Y can be a straight chain or branched C1-C6 alkyl group or a straight chain or branched C1-C6 alkyl ether group wherein the carbon atoms of the alkyl group or alkyl ether group each independently comprise linked hydrogen, deuterium or fluorine atoms. In some embodiments, Y is methyl, ethyl, n-propyl, isopropyl, n-butyl, isobutyl, sec-butyl or tert-butyl. The group R can be a straight chain or branched C1-C6 alkyl group or a substituted or unsubstituted phenyl group, wherein the carbon atoms of the alkyl group or phenyl group each independently comprise linked hydrogen, deuterium or fluorine atoms. In some embodiments, the N-substituted amino acid ester is the ester (e.g. methyl or ethyl) of sarcosine, which is an ester of N-methyl glycine.

Every possible permutation of ¹⁵N or ¹³C labeled glycine is commercially available. Likewise, other natural amino acids are commercially available with one or more incorporated heavy atom isotopes. Because glycine, and other amino acids, comprising one or more heavy atom isotopes are commercially available, these amino acids can be easily incorporated into the procedure for the production of N-substituted piperazines. The amino acids comprising heavy atom isotopes can be N-protected using procedures well-known in peptide chemistry. For example, the amino acids can be N-protected with a 9-fluorenylmethoxycarbonyl (Fmoc) group or a t-boc group. Furthermore, the amino acids comprising heavy atom isotopes can be N-alkylated and converted to an ester of the amino acid using well-known procedures. Accordingly, heavy atom isotope containing starting materials for the preparation of N-substituted piperazines, as described herein, are either commercially available, or can be easily prepared from commercially available amino acids using no more than routine experimentation.

According to the method, the two amino acids can be condensed to thereby produce an N-protected peptide dimer as an ester. The N-protected peptide dimer ester can comprise one or more heavy atom isotopes via the incorporation of the one or more amino acids comprising one or more heavy atom isotopes. The N-protected peptide dimer ester can comprise one heavy atom isotope, two heavy atom isotopes, three heavy atom isotopes, four heavy atom isotopes, five heavy atom isotopes or six heavy atom isotopes. The N-protected peptide dimer can comprise greater than six heavy atom isotopes. The N-protected peptide dimer ester can have the general formula: 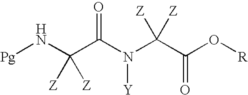
wherein Pg, R, Y and Z are previously defined.

According to the method, the N-protected peptide dimer ester can then be cyclized to form a 6-membered cyclic dione. Cyclization proceeds by removing the N-protecting group of the N-protected peptide dimer ester and driving the reaction of the deprotected amine with the ester group. The reaction can be carried out under basic conditions and can be heated to speed production of the product. The product of the cyclization can have the general formula: 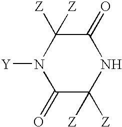
wherein Y and Z are previously defined.

According to the method, the ketone groups of the cyclic dione can then be reduced to form the desired N-substituted piperazine comprising one or more heavy atom isotopes. The reduction can be performed using a reducing agent, such as lithium aluminum hydride (LAH) or Red-Al (Sigma-Aldrich). The product, in some embodiments being a volatile oil, can be optionally temporarily modified (e.g. protected) to aid in isolation. Because piperazine comprises two basic nitrogen atoms, the product can, in some embodiments, be isolated as a mono or bis-acid salt. For example, the N-substituted piperazine comprising one or more heavy atom isotopes can be isolated as a mono-TFA salt, a mono-HCl salt, a bis-TFA salt or a bis-HCl salt.

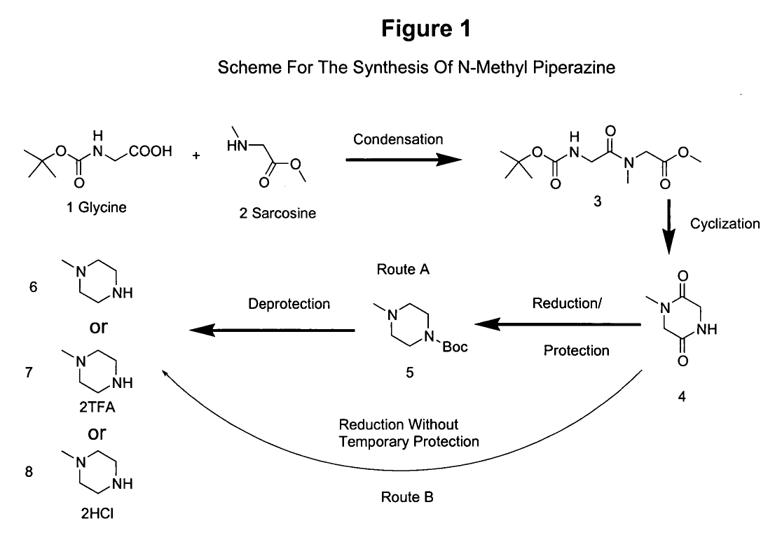

FIG. 1 illustrates the application of the aforementioned general procedure to the production of N-methyl piperazine. Examples 1-4 describe the application of the illustrated procedure to the production of three different N-methyl piperazines each comprising 1-3 heavy atom isotopes.

With reference to FIG. 1 and Examples 1-4, t-boc protected glycine (1) is condensed with sarcosine methyl ester (2) to thereby produce the dipeptide (3). The t-boc group is removed and the dipeptide is cyclized to the cyclic dione (4). The ketone groups of the dione are then reduced to produce N-methyl piperazine. The N-methyl piperazine product can either be transiently protected (5) or can be obtained directly from the reduction (6). The product can also be obtained as a salt of an acid (e.g. TFA salt (1) or HCl (8)).

In summary, a wide variety of N-substituted piperazine compounds, unlabeled or labeled with one or more heavy atom isotopes, can be produced by the aforementioned process. Consequently, the present invention contemplates all possible isotopically enriched N-substituted piperazine compound comprising one or more heavy atom isotopes of the general formula: 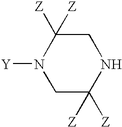
including all possible salt forms thereof, wherein Y and Z are previously defined.
II. Preparation of N-Substituted Piperazine Acetic Acids Comprising Heavy Atom Isotopes

In some embodiments, this invention pertains to methods for the production of isotopically enriched N-substituted piperazine acetic as well as the isotopically enriched N-substituted piperazine acetic acids. In some embodiments, an N-substituted piperazine can be reacted with a halo acetic acid moiety comprising one or more heavy atom isotopes. In this context, halo refers to the halogens, chlorine, bromine and iodine. In still some other embodiments, an N-substituted piperazine comprising one or more heavy atom isotopes can be reacted with a halo acetic acid moiety. In some other embodiments, an N-substituted piperazine comprising one or more heavy atom isotopes can be reacted with a halo acetic acid moiety comprising one or more heavy atom isotopes. Accordingly, the heavy atom isotopes found in the product N-substituted piperazine acetic acids can be introduced by way of the piperazine, by way of the halo acetic acid moiety or by way of both the piperazine and the halo acetic acid moiety. As will be discussed in more detail below, ¹⁸O can also be introduced into the carboxylic acid moiety of an N-substituted piperazine acetic acid by way of exchange with H₂¹⁸O.

Numerous light (by “light” we mean that the compound is not isotopically enriched with one or more heavy atom isotopes) N-substituted piperazines (e.g. N-methyl and N-ethyl piperazine) are commercially available. Furthermore, Section I above describes the preparation of N-substituted piperazine comprising one or more heavy atoms from commercially available amino acids. Both light and heavy (by “heavy” we mean that the compound has been isotopically enriched with one or more heavy atom isotopes) N-substituted piperazine can be used to produce the N-substituted piperazine acetic acids comprising one or more heavy atom isotopes.

Numerous light and heavy halo acetic acid moieties are commercially available. The halo acetic acid moiety to be reacted with the N-substituted piperazine can be purchased as the carboxylic acid or as an ester of the carboxylic acid (e.g. the methyl ester, ethyl ester or phenyl ester). If only the carboxylic acid is available and the ester is desired, the ester can be prepared using well-known esterification methods. If only the ester is available and the carboxylic acid is desired, the ester can be hydrolyzed to produce the carboxylic acid. Either the carboxylic acid or the ester can be used in the alkylation reaction provided that an additional equivalent of base is required if the carboxylic acid is used. If the ester is used to perform the alkylation, the product ester can be hydrolyzed to produce the N-substituted piperazine acetic acid. General structures for the carboxylic acid and the ester compounds that can be used to alkylate N-substituted piperazines are: 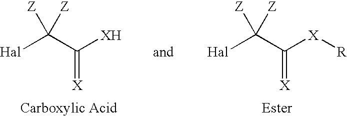
wherein Z and R are defined above. Hal is a halogen (Cl, Br or I) and X is oxygen (O) or sulfur (S). In some embodiments, X is ¹⁶O or ¹⁸O. One or more of the atoms of the halo acetic acid compound can be a heavy atom isotope.

The alkylation of an N-substituted piperazine with a halo acetic acid moiety proceeds under basic conditions. The base need only be strong enough to deprotonate piperazine but can be selected to not substantially react with the halo acetic acid moiety. In some embodiments, two or more equivalents of N-substituted piperazine can be used, as N-substituted piperazine is a base. If it is desirable to use only one equivalent of N-substituted piperazine (for example, when the N-substituted piperazine is labeled with one or more heavy atom isotopes and is therefore valuable), other bases can be used. Suitable bases include, but are not limited to, hindered bases such as triethylamine (Et₃N) and diisopropylethyamine (DIEPA). Other suitable bases in sodium carbonate and potassium carbonate. Hindered bases are a good choice because they do not react substantially with the halo acetic acid moiety.

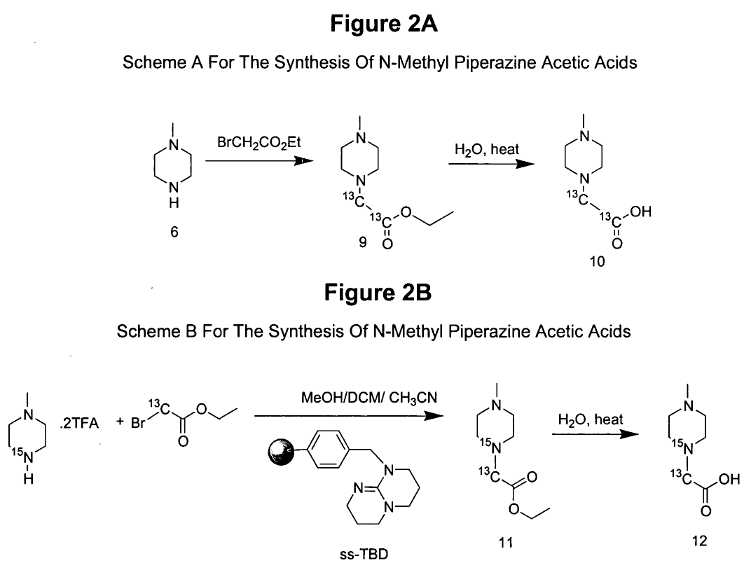

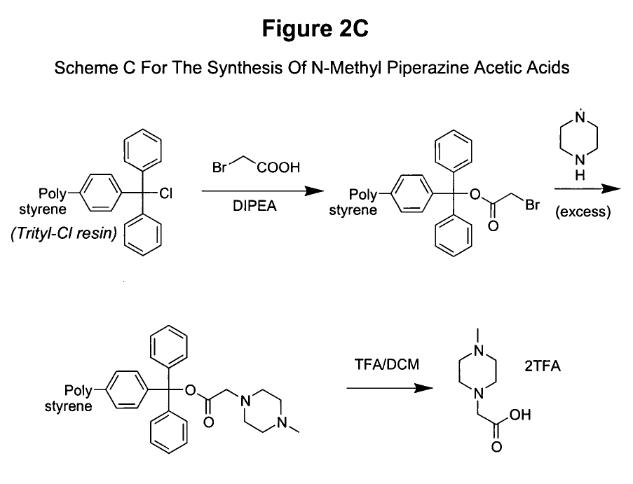

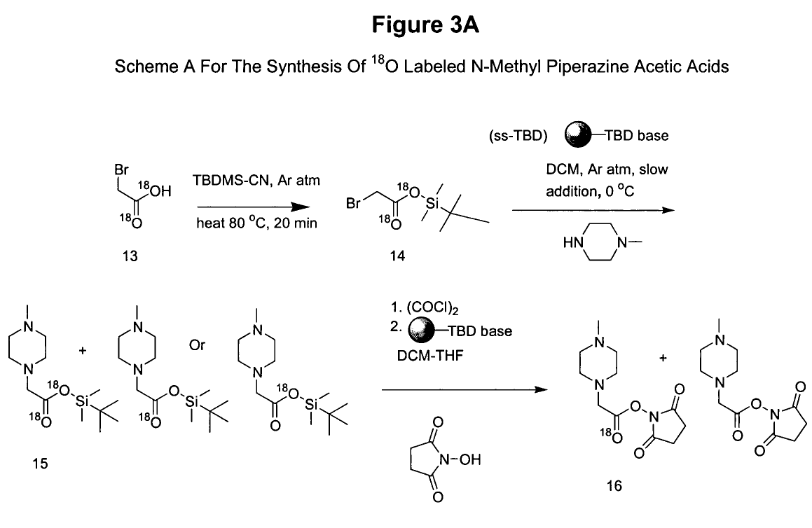

A solid phase base, such as 1,5,7-Triazabicyclo[4.4.0]dec-5-ene (TBD) bound to polystyrene crosslinked with 2% DVB, Capacity (base): ˜2.6 mmol/g (ss-TBD, Fluka, P/N 90603) can also be used (See FIG. 2B). A solid phase base has the advantage that it is easily, and completely, removed from the product by filtration once the alkylation reaction has been completed. Accordingly, the resulting product is not contaminated with salt of the base.

If the carboxylic acid is used to alkylate the N-substituted piperazine, the reaction can produce a product of the general formula: 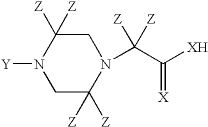
or a salt thereof, wherein X, Y and Z have been previously defined. One or more atoms of the N-substituted piperazine acetic acid can be a heavy atom isotope.

If the ester is used to alkylate the N-substituted piperazine, the reaction will produce an ester of the general formula: 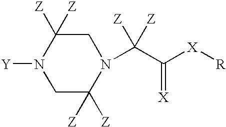
or a salt thereof, wherein R, X, Y and Z have been previously defined. One or more atoms of the N-substituted piperazine acetic acid ester can be a heavy atom isotope. The N-substituted piperazine acetic acid ester can be converted to the N-substituted piperazine acetic acid of general formula: 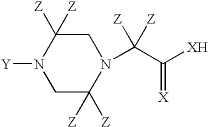
by hydrolysis of the ester. Depending on the state of protonation of the ester, it may or may not be necessary to add base to aqueous solution to perform the hydrolysis because piperazine is basic (unless neutralized by acid). Accordingly, base can be added as required to induce the hydrolysis of the ester to the carboxylic acid, but in some embodiments it will not be required. Hydrolysis can also be performed under aqueous acidic conditions.

N-substituted piperazine acetic acid is zwitterionic. Because it comprises a carboxylic acid group (or thio acid group) and two basic nitrogen atoms, it can exist in at least four different forms. It can exist completely deprotonated as its carboxylate anion. It can exist as its mono protonated zwitterion. It can exist as a monobasic salt (e.g. mono-TFA or mono-HCl salt). It can also exist as its dibasic salt (e.g. bis-TFA or bis-HCl salt). The state of protonation of the product is a function of the conditions under which it was isolated. All protonation states of N-substituted piperazine acetic acid are contemplated as embodiments of the present invention.

With reference to FIGS. 2A and 2B, as well as Examples 5 and 6, respectively, the production of two different isotopically enriched N-methyl piperazine acetic acid compounds is described. In FIG. 2A and Example 5, two equivalents of commercially available unlabeled N-methyl piperazine is reacted with ethyl bromoacetate to produce a N-methyl piperazine acetic acid compound comprising two ¹³C atoms. Because N-methyl piperazine is basic, hydrolysis of the ethyl ester proceeded by merely heating the compound in an aqueous solution.

With reference to FIG. 2B and Example 6, the starting piperazine is a bis-TFA salt of ¹³N labeled N-methyl piperazine. Acid salts of the piperazine base can be alkylated so long as sufficient base is added to the reaction to deprotonate piperazine. In this example, the ethyl bromoacetate is ¹³C labeled. Because both the piperazine and acetic acid reactants comprise heavy atom isotopes, a solid phase base was chosen so that only one equivalent of each reactant was required to produce the product. As was observed with Example 5, hydrolysis of the ethyl ester proceeded by mere heating the compound in an aqueous solution.

In some other embodiments, the N-substituted piperazine acetic acid can be assembled on a solid support. According to the method and with reference to FIG. 2C, the halo acetic acid moiety, as a carboxylic acid, can be reached with trityl chloride resin to thereby produce a support bound halo acetic acid. The support bound halo acetic acid can then be treated with the desired N-substituted piperazine (e.g. N-methyl piperazine) under basic conditions to thereby produce the N-substituted piperazine acetic acid. Isotopically enriched N-methyl piperazine and halo acetic acid moieties can be used, including ¹⁸O labeled compounds although ¹⁸O labeling can involve special considerations and is discussed in more detail below.

In accordance with the aforementioned discussion, a heavy atom isotope can be incorporated at virtually any position of the N-substituted piperazine acetic acid, including ¹⁸O incorporation that will be discussed in more detail below. Consequently, the present invention contemplates all possible isotopically enriched N-substituted piperazine acetic acids comprising one or more heavy atom isotopes of the general formula: 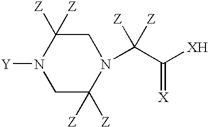
including all possible salt forms thereof.
III. Incorporation of ¹⁸O into N-Substituted Piperazine Acetic Acids

In some embodiments, this invention pertains to methods for the incorporation of ¹⁸O into N-substituted piperazine acetic acids as well as to the ¹⁸O labeled N-substituted piperazine acetic acids themselves. In some embodiments, incorporation of ¹⁸O is not substantially different as compared with the methods described for the preparation of isotopically labeled N-substituted piperazine acetic acids in Section II, above. In some other embodiments, incorporation of ¹⁸O is substantially different and takes advantage of the very caveat that creates some concern about the methods previously discussed.

The caveat with respect to the preparation of ¹⁸O labeled N-substituted piperazine acetic acids lies with the exchange of ¹⁸O [symbol] ¹⁶O that can occur between unlabeled water (H₂¹⁶O) and the ¹⁸O of a heavy carboxylic acid group. A carboxylic acid group is inherently acidic. Acid can catalyze the exchange of the oxygen atoms of a carboxylic acid group and water, such as residual water in a sample or water used in a reaction (e.g. hydrolysis of an ester). Consequently, whenever ¹⁸O labeled N-substituted piperazine acetic acids were desired, one of two different synthetic routes was chosen.

In some embodiments, the ¹⁸O labeled N-substituted piperazine acetic acid was obtained by alkylation with an appropriately ¹⁸O labeled halo acetic acid moiety. The procedure is essentially as outlined in Section II, above except that an acid labile ester of the halo acetic acid was used in the alkylation reaction. In some embodiments, the halo acetic acid moiety comprised the formula: 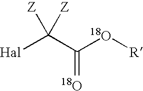
wherein Hal is previously defined and R′ is an acid labile ester group, including but not limited to tert-butyldimethylsilyl or t-boc.

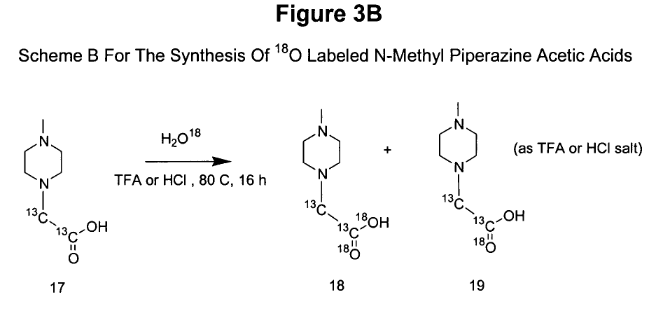

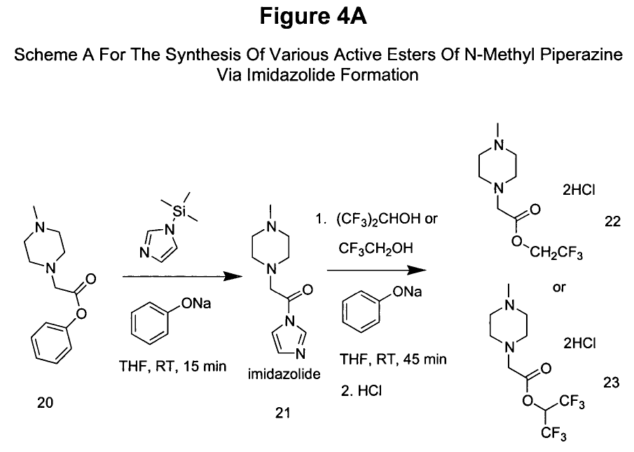

With reference to FIG. 3A and Example 8, the tert-butyldimethylsilyl (TBDMS) ester of (¹⁸O)₂ bromoacetic acid (14) was used in the alkylation reaction. This ester was prepared using ¹⁸O labeled bromoacetic acid (13), obtained as a custom order from Cambridge Isotope Laboratory, Inc., and TBDMS-CN. The TBDMS ester of N-methyl piperazine acetic acid (15) was the product of the alkylation with N-methyl piperazine. The TBDMS ester was selected so that it could be converted to the acid chloride with, for example, oxalyl chloride thereby avoiding the requirement for any water and the possible exchange of ¹⁸O with ¹⁶O. In the presence of solid phase base (ss-TBD) and N-hydroxysuccinimide (NHS), the acid chloride was converted to the NHS ester (16). If the carboxylic acid is desired, instead of the active ester, the TBDMS ester could be converted to the carboxylic acid by treatment with an anhydrous acid such as TFA. Accordingly, aqueous treatment that might lead to ¹⁸O [symbol] ¹⁶O exchange, can be avoided whether to active ester or the carboxylic acid is desired.

In some other embodiments, the alkylation to produce N-substituted piperazine acetic acid proceeded as described in Section II, above and the ¹⁸O was later incorporated. With reference to FIG. 3B and Example 9, it was found that ¹⁸O could be incorporated into the carboxylic acid group of any N-substituted piperazine acetic acid by treatment of the N-substituted piperazine acetic acid with H₂¹⁸O under acidic conditions. For example and with reference to FIG. 3B, an isotopically enriched N-methyl piperazine acetic acid (17) lacking ¹⁸O, used to produce the 114 labeling reagent, was treated with H₂¹⁸O and either HCl or TFA to thereby produce the TFA or HCl salt of the ¹⁸O isotopically enriched N-methyl piperazine acetic acid (18) and (19).

Furthermore, the isotopic purity of the product could be increased by repeated cycles of treatment with H₂¹⁸0 under acidic conditions. The higher the state of enrichment of the H₂¹⁸O, the fewer cycles required to produce highly ¹⁸O enriched N-substituted piperazine acetic acid. When H₂¹⁸O of 99% purity was used, the isotopic enrichment of N-substituted piperazine acetic acid was typically 96% after two cycles. One or more additional cycles can be performed to further increase the ¹⁸O isotopic content of the product N-substituted piperazine acetic acids. Because this exchange was performed under acidic conditions, the product was easily isolated as the bis-acid salt of N-substituted piperazine acetic acid (e.g. the bis-TFA or bis-HCl salt).

Consequently, the present invention contemplates all possible isotopically enriched N-substituted piperazine acetic acids comprising one or more heavy atom isotopes of the general formula: 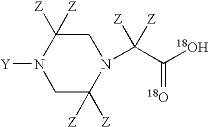
including all possible salt forms thereof.
IV. Preparation of Various Active Esters of N-Substituted Piperazine Acetic Acid

In some embodiments, this invention pertains to methods for the preparation of active esters of N-substituted piperazine acetic acid, including isotopically enriched versions thereof, as well as the N-substituted piperazine acetic acid esters themselves, and isotopically enriched versions thereof. The active ester can be any active ester. In some embodiments, the active ester can be formed using an alcohol or thiol of the following formula: 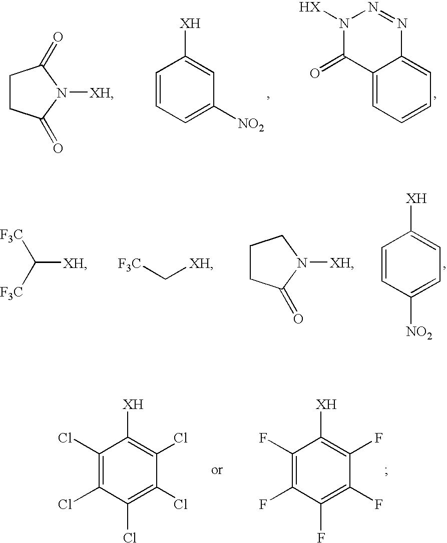
wherein X is O or S, but preferably O. In some other embodiments, the active ester can be formed using an alcohol or thiol of the following formula: 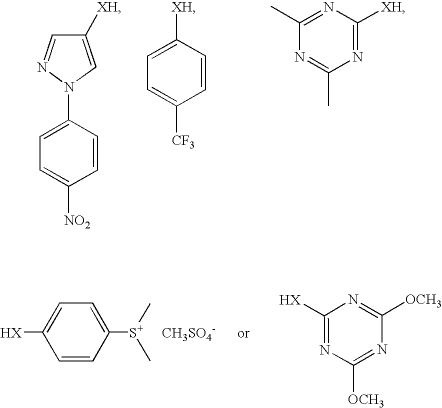
wherein X is O or S, but preferably O.

In some embodiments, the active ester can be prepared through an intermediary imidazolide. According to this method, an N-substituted piperazine acetic acid ester, including isotopically enriched versions thereof, can be converted to the imidazolide. The imidazolide so prepared can then be reacted with the alcohol of choice to thereby produce the active ester of the selected alcohol.

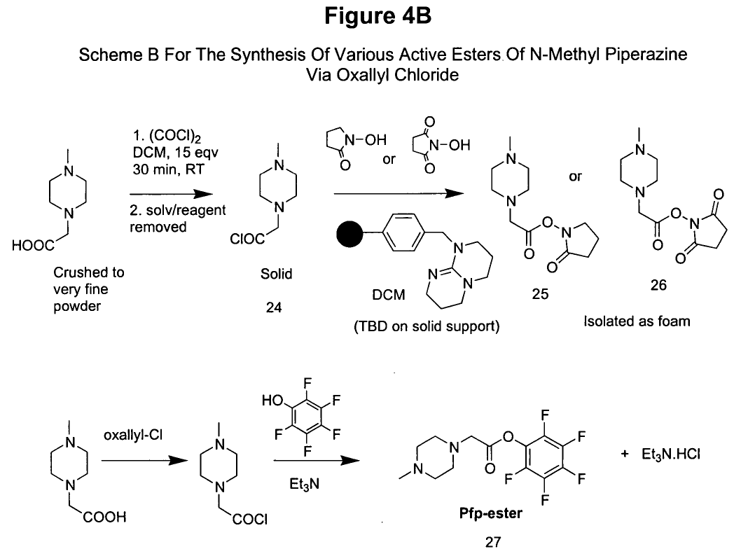

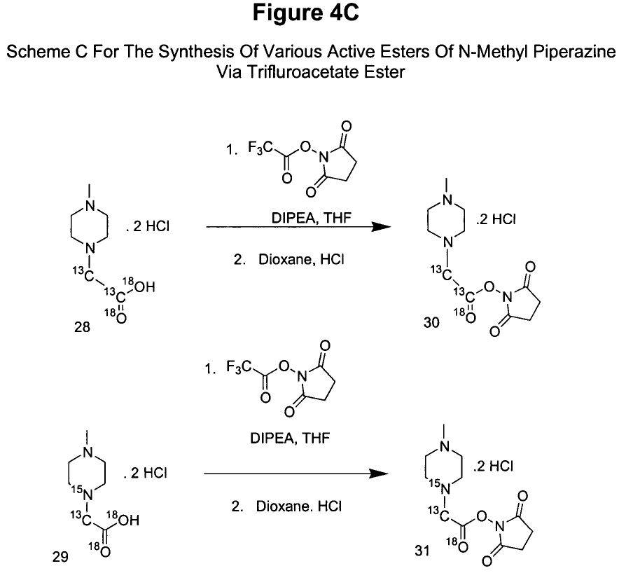

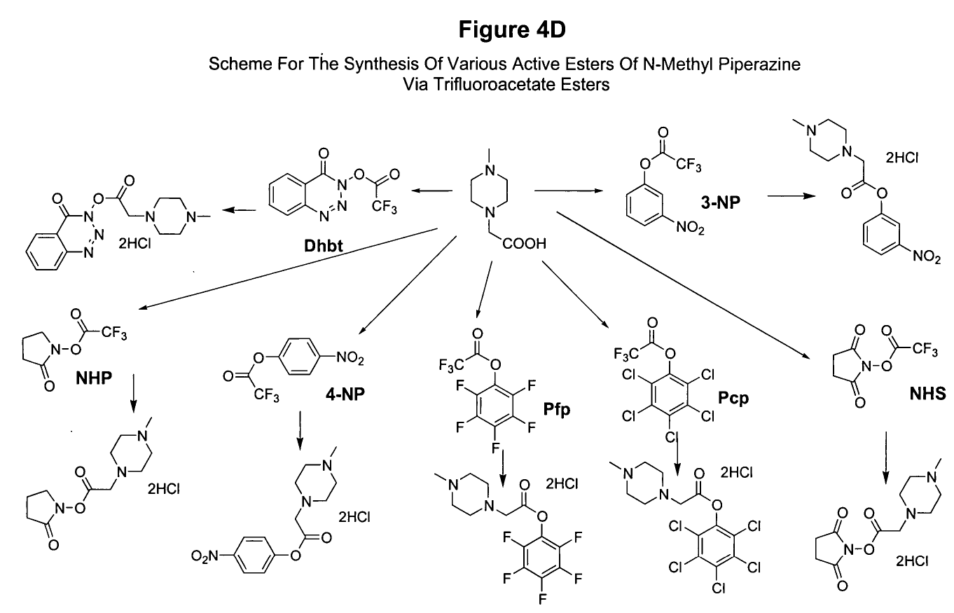

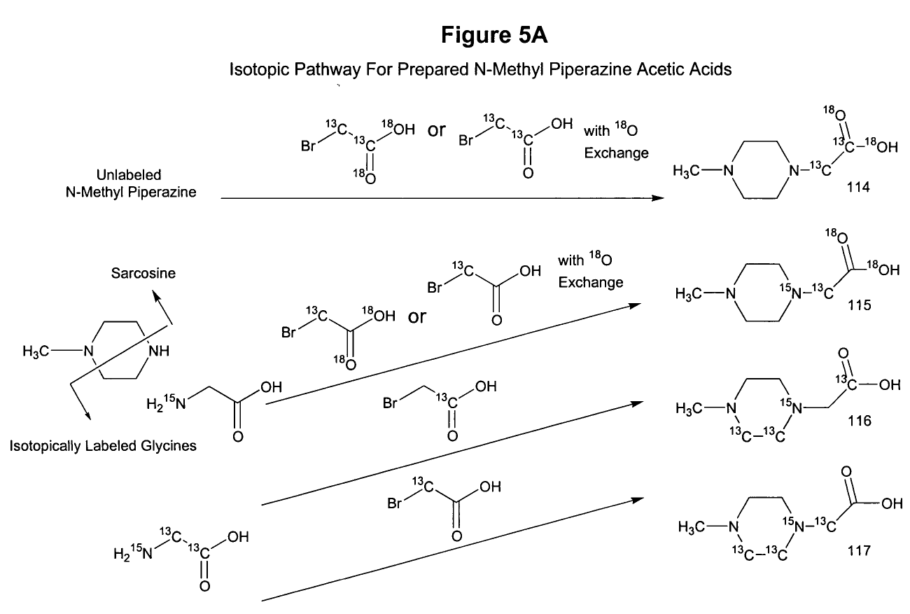

With reference to FIG. 4A and Example 10, this procedure was used to prepare active esters of 2,2,2-trifluorethanol and 1,1,1,3,3,3-hexafluoro-2-propanol. According to the figure and the example, the phenyl ester of N-methyl piperazine acetic acid (20) was treated with trimethyl silyl imidizole (TMS-imidizole) and sodium phenoxide to form the imidazolide of N-methyl piperazine acetic acid (21). The imidazolide (21) was then reacted with either 2,2,2-trifluorethanol or 1,1,1,3,3,3-hexafluoro-2-propanol (HFI—OH) to produce the desired active ester of N-methyl piperazine acetic acid (22) or (23), respectively as a bis-acid salt.

In some other embodiments, the active ester can be prepared by conversion of the N-substituted piperazine acetic acid, including isotopically enriched versions thereof, to an acid chloride followed by subsequent reaction of the acid chloride with the alcohol of choice to thereby produce the active ester of the selected alcohol.

With reference to FIG. 4B and Example 11, the preparation of the NHS and NHP esters of N-methyl piperazine acetic acid are illustrated using this general procedure. According to the figure and the example, N-methyl piperazine acetic acid is treated with oxalyl chloride to produce the acid chloride (24). The acid chloride is then treated with either of NHP or NHS and solid phase base to thereby produce the active ester (25) or (26), respectively as the free piperazine base (not as an acid salt).

FIG. 4B also illustrates the application of oxalyl chloride to the production of the pentafluorophenyl (Pfp) ester (27) wherein a solution phase base (e.g. triethylamine) is used. The reaction proceeded well with the solution phase base but the hydrochloride salt of the base proved difficult to remove. Application of the solid phase base avoids this caveat.

In still some other embodiments, the active ester can be prepared by treatment of the N-substituted piperazine acetic acid, including isotopically enriched versions thereof, with a trihalooacetate ester of the alcohol that is desired to form the active ester of the N-substituted piperazine acetic acid. In this context, halo refers to fluorine, chlorine, bromine and iodine but preferably to fluorine and chlorine. The trihalooacetate ester has the general formula: 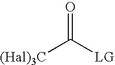
wherein Hal refers to a halogen (fluorine, chlorine, bromine or iodine) and LG refers to the leaving group alcohol. The leaving group (LG) of the trihaloacetate esters can have the following general formula: 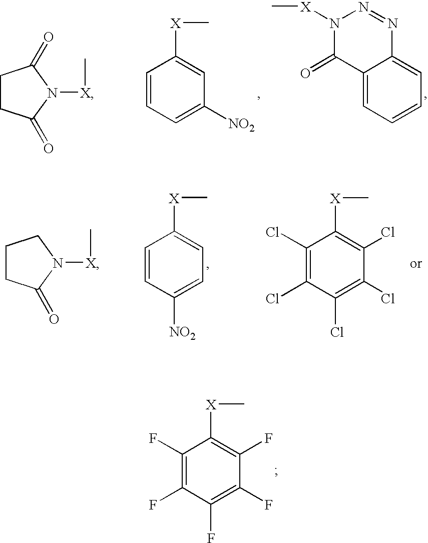
wherein X is O or S, but preferably O. Active esters of N-methyl piperazine acetic acid comprising these leaving groups (LG) were successfully prepared using the identified trifluoroacetate esters (where X is O).

This procedure can be applied to the N-substituted piperazine acetic acids whether they are the acid salt or the zwitterion form. The N-substituted piperazine acetic acids can be reacted with the triholoacetate ester of the alcohol to thereby produce the active ester of the N-substituted piperazine acetic acid. A base that can deprotonate the basic nitrogen atoms of piperazine ring can be added to the reaction as need to induce formation of the product when the starting material is an acid salt of N-substituted piperazine acetic acid. The active ester of the N-substituted piperazine acetic acid can itself be isolated as the mono-acid salt or the di-acid salt. (e.g. the mono-TFA salt, the mono HCl salt, the bis-TFA salt or the bis-HCl salt.). When the trihalooacetate ester is reacted with an N-substituted piperazine acetic acid the product can be: 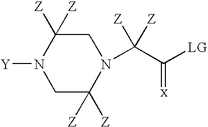
or a salt thereof, wherein X, Y and Z are previously defined. The group LG is the leaving group of the active ester that is displaced by the reactive group of an analyte to be labeled; in essence the leaving group is the alcohol used to form the active ester.

Certain trihaloacetate esters are commercially available. For example, the trifluoracetate esters of pentafluorphenol and 4-nitrophenol can be purchased from commercial sources. However, the others can be obtained by reacting the desired alcohol with trihaloacetic anhydride. With reference to Table 1, below, the trifluoroacetate esters of pentachlorophenol (Pcp), 3-hydroxy-1,2,3-benzotriazine-4(3H)-one (Dhbt), NHS, 3-nitrophenol (3-NP) and N-hydroxypyrrolidinone (NHP) were prepared by reacting the respective alcohol with trifluoracetic anhydride. The general procedure for such reactions can be found in Example 12. Other alcohols that can be used to produce trihaloacetate esters suitable for the formation of other active esters can also be used.

FIG. 4C illustrates the production of the 114 and 115 labeling reagents as the NHS ester. Accordingly, the procedure was successfully applied to the production of isotopically enriched active esters of N-substituted piperazine acetic acids. These active ester reagents were produced as the bis-HCl salts from the bis-HCl salts of the piperazine base.

FIG. 4D illustrates the production of numerous other active esters of N-methyl piperazine acetic acid that were produced using this generic process. As will be appreciated by the ordinary practitioner, this procedure is generic and robust and can be applied to the production of numerous other active esters of a plethora of N-substituted piperazine acetic acid derivatives.

V. Isotope Incorporation Pathway for the Preparation of a Set of Isobaric Labeling Reagents

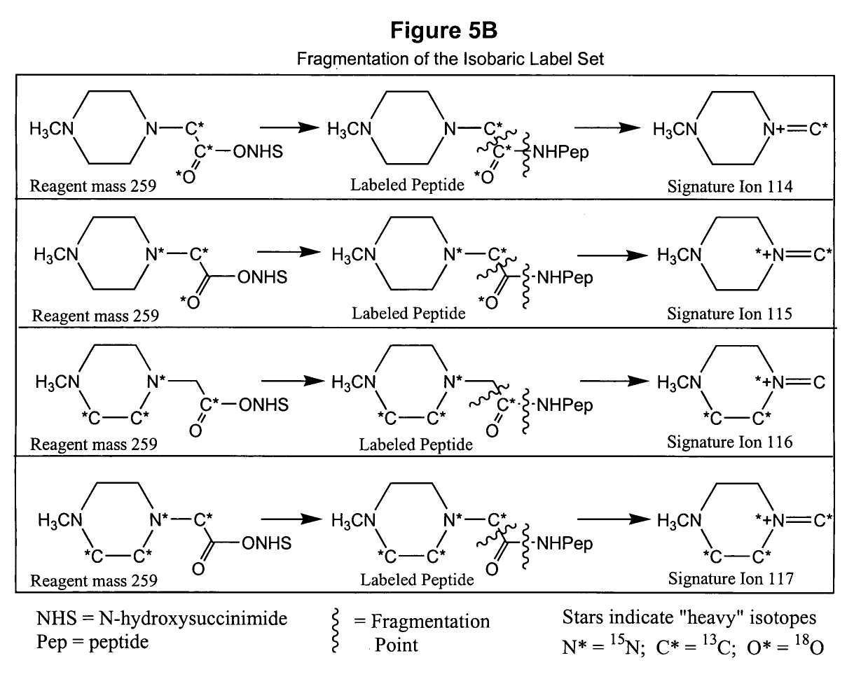

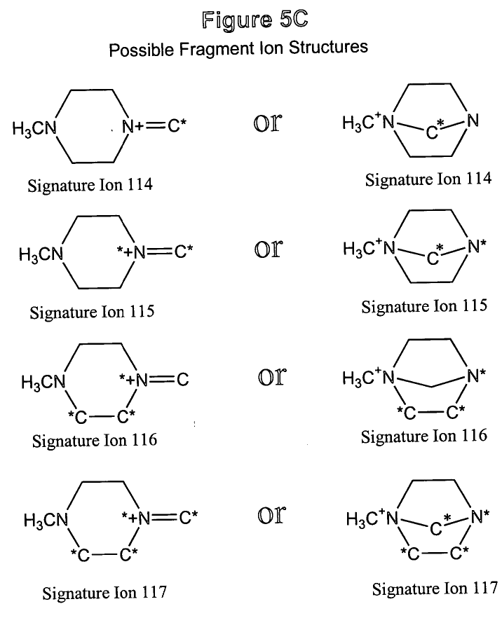

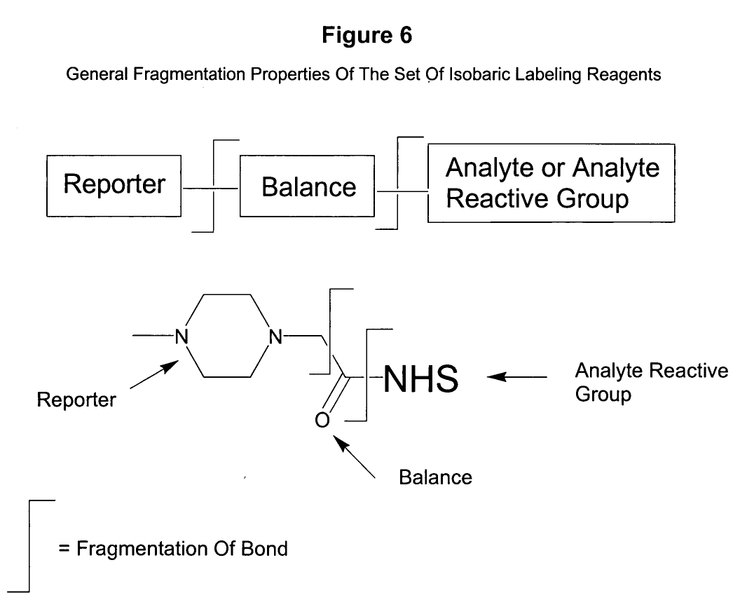

FIG. 5A illustrates the general pathway taken to the production of a set of four isobaric labeling reagents identified as 114, 115, 116 and 117. These designations are based upon the “signature ion” each reagent produces upon fragmentation in a mass spectrometer (FIG. 5B). The “signature ion” can be used to deconvolute information associated with different samples in a multiplex assay.

The pathways illustrated in FIG. 5A utilize the procedures set forth above for the production of N-substituted piperazine acetic acids, and active esters thereof. In particular, suitable isotopically labeled glycines were used in the preparation of suitable isotopically labeled N-substituted piperazines (i.e. N-methyl piperazines). The labeled and unlabeled N-methyl piperazines can be treated with isotopically labeled bromoacetic acid derivatives, with or without subsequent ¹⁸O enrichment, to thereby produce the N-methyl piperazine acetic acid compounds of desired structure. These suitably labeled N-methyl piperazine acetic acid compounds were used as labeling reagents; in the present case by conversion to an active ester (e.g. NHS ester) for coupling with analytes such as peptides.

VI. State of Isotopic Enrichment

The various N-substituted piperazines, N-substituted piperazine acetic acids and active esters of N-substituted piperazine acetic acid can be prepared with starting materials of greater than 80 percent isotopic purity for each heavy atom isotope. The isotopic purity can be greater than 93 percent for each heavy atom isotope in some starting materials. In other starting materials the isotopic purity can be greater than 96 percent for each heavy atom isotope. In still other starting materials the isotopic purity can be greater than 98 percent for each heavy atom isotope. When performing an ¹⁶O to ¹⁸O exchange, it was possible to routinely obtain carboxylic acid groups of 96 or greater percent isotopic purity (per oxygen atom) of the heavy atom isotope.

Because, with the exception of ¹⁸O, which can be exchanged with ¹⁶O in certain cases, the isotope purity and composition of starting materials will translate directly into the isotopic purity of the products. Moreover, for ¹⁸O, it has been shown that isotopic purity of greater than 96 percent (per atom) can be achieved using the methods described herein. Accordingly, in some embodiments, this invention pertains to N-substituted piperazines, N-substituted piperazine acetic acids and/or active esters of N-substituted piperazine acetic acid having an isotopic purity of at least 80 percent for each heavy atom isotope. In some other embodiments, this invention pertains to N-substituted piperazines, N-substituted piperazine acetic acids and/or active esters of N-substituted piperazine acetic acid having an isotopic purity of at least 93 percent for each heavy atom isotope. In still some other embodiments, this invention pertains to N-substituted piperazines, N-substituted piperazine acetic acids and/or active esters of N-substituted piperazine acetic acid having an isotopic purity of at least 96 percent for each heavy atom isotope. In yet some other embodiments, this invention pertains to N-substituted piperazines, N-substituted piperazine acetic acids and/or active esters of N-substituted piperazine acetic acid having an isotopic purity of at least 98 percent for each heavy atom isotope.

VII. Labeling of Analytes with Active Esters of N-Substituted Piperazine Acetic Acids:

Generally, active esters of N-substituted piperazine acetic acid are electrophilic and can be reacted with nucleophilic groups of the analyte to thereby produce labeled analytes. In some embodiments, the labeling reagent can be support bound. Active esters can react with thiol groups, and to a lesser extent with hydroxyl groups. However, active esters are particularly well suited for reaction with amine groups.

The active esters of N-substituted piperazine acetic acid can be reacted with an analyte in solution to thereby produce a labeled analyte. The solution used to effect labeling can be selected depending upon the nature of the analyte and the labeling reagent. Conditions can be selected such that the labeling reagent and/or the analyte are soluble, or at least partially soluble. Because analytes can be biomolecules, the labeling conditions can be selected to be aqueous. Aqueous conditions can include organic modifiers that increase the solubility of the labeling reagent and/or analyte. Organic modifiers can be water miscible and can include, but are not limited to, alcohols (such as methanol, ethanol, n-propanol, iso-propanol or n-butanol or t-butanol) ethers (such as tetrahydrofuran or dioxane) acetonitrile (ACN), N,N-dimethylformamide (DMF), N-Methyl pyrrolidine (NMP) and the like. The reactions can be performed under conditions of preferred ionic strength and/or pH.

Generally, the pH of aqueous solutions can be modulated with a buffer. The pH can be adjusted to be within the range of 4-10. The pH can be adjusted to be outside of this range. The basicity of non-aqueous reactions can be modulated by the addition of non-nucleophilic organic bases. Non-limiting examples of suitable bases include N-methylmorpholine (NMM), triethylamine (Et₃N) and N,N-diisopropylethylamine (DIPEA). Alternatively, the pH can be modulated using biological buffers such as (N-[2-hydroxyethyl]piperazine-N′-[2-ethanesulfonic acid) (HEPES) or 4-morpholineethane-sulfonic acid (MES). Inorganic buffers such as sodium bicarbonate or sodium carbonate can also be used to control pH. Non-nucleophilic buffers can be used so that the buffers do not react with the labeling reagent.

All four of the isobaric labeling reagents of N-methylpiperazine acetic acid (114, 115, 116 and 117; See FIG. 5B) were produced as NHS esters. These reagents comprise an exemplary set of isobaric labeling reagents. Two or more of the reagents were used to label peptides, including peptides (analytes) obtained from digested protein (See: Examples 15 & 16). The set of reagents (two or more of them), were shown to be suitable for the multiplex analysis, including proteome analysis of yeast using the general procedures as described in copending and co-owned U.S. patent application Ser. No. 10/765,458, incorporated herein by reference.

Because the reagents are the labeling reagents are non-polymeric, low molecular weight isobaric compounds, there is no significant signal splitting in either the MS or MS/MS modes that might otherwise complicate the spectra.

VIII. Fragmentation by Dissociative Energy Levels:

It is well accepted that bonds can fragment as a result of the processes occurring in a mass spectrometer. Moreover, bond fragmentation can be induced in a mass spectrometer by subjecting ions to dissociative energy levels. For example, the dissociative energy levels can be produced in a mass spectrometer by collision-induced dissociation (CID). Those of ordinary skill in the art of mass spectrometry will appreciate that other exemplary techniques for imposing dissociative energy levels that cause fragmentation include, but are not limited to, photo dissociation, electron capture and surface induced dissociation.

The process of fragmenting bonds by collision-induced dissociation involves increasing the kinetic energy state of selected ions, through collision with an inert gas, to a point where bond fragmentation occurs. For example, kinetic energy can be transferred by collision with an inert gas (such as nitrogen, helium or argon) in a collision cell. The amount of kinetic energy that can be transferred to the ions is proportional to the number of gas molecules that are allowed to enter the collision cell. When more gas molecules are present, a greater amount of kinetic energy can be transferred to the selected ions, and less kinetic energy is transferred when there are fewer gas molecules present.

It is therefore clear that the dissociative energy level in a mass spectrometer can be controlled. It is also well accepted that certain bonds are more labile than other bonds. The lability of the bonds in an analyte or the reporter/linker moiety depends upon the nature of the analyte or the reporter/linker moiety. Accordingly, the dissociative energy levels can be adjusted so that the analytes and/or the labels (e.g. the reporter/linker combinations) can be fragmented in a manner that is determinable. One of skill in the art will appreciate how to make such routine adjustments to the components of a mass spectrometer to thereby achieve the appropriate level of dissociative energy to thereby fragment at least a portion of ions of labeled analytes into ionized reporter moieties and daughter fragment ions.

For example, dissociative energy can be applied to ions that are selected/isolated from the first mass analysis. In a tandem mass spectrometer, the extracted ions can be subjected to dissociative energy levels and then transferred to a second mass analyzer. The selected ions can have a selected mass to charge ratio. The mass to charge ratio can be within a range of mass to charge ratios depending upon the characteristics of the mass spectrometer. When collision induced dissociation is used, the ions can be transferred from the first to the second mass analyzer by passing them through a collision cell where the dissociative energy can be applied to thereby produce fragment ions. For example the ions sent to the second mass analyzer for analysis can include some, or a portion, of the remaining (unfragmented) selected ions, as well as reporter ions (signature ions) and daughter fragment ions of the labeled analyte.

IX. Signature Ions:

FIG. 5C is an illustration of two possible structures for the signature ions of the 114, 115, 116 and 117 labeling reagents. Because there are alternative chemical structures that might be correct, reference is made to the molecular formulas of the signature ions. The molecular formula of the light signature ion is C₆H₁₃N₂⁺. The molecular formulas of the isotopically enriched 114, 115, 116 and 117 signature ions respectively are: ¹³CC₅H₁₃N₂⁺, ¹³CC₅H₁₃¹⁵NN⁺, ¹³C₂C₄H₁₃¹⁵NN⁺ and ¹³C₃C₃H₁₃¹⁵NN⁺.

X. General Properties of the Isobaric Labeling Reagents when Fragmented

With reference to FIG. 6, the general properties of the isobaric labeling reagents when fragmented are illustrated. The isobaric labeling reagents can be considered to comprise a reporter, a balance and an analyte reactive group. Once the analyte has been labeled, the analyte can be linked directly to the balance.

As illustrated, there is a bond that links the reporter to the balance and a bond that links the balance to the analyte (or the analyte reactive group). When subjected to dissociative energy levels these bonds can be fragmented. In some embodiments, fragmentation of one of the bonds will result in fragmentation of the other bond. In this way, little or no partially labeled analyte is observed in the MS/MS spectrum. This results in a cleaner spectrum. When the label is fragmented, the reporter will produce the signature ion and the balance can be selected to result in a neutral loss or it can also produce a fragment ion. If the balance results in neutral loss (uncharged fragment) the fragment is not observed in the MS/MS spectrum. This results in a cleaner spectrum.

As illustrated in FIG. 6, each of the reagents of the isobaric set of N-methyl piperazine labeling reagents comprises a reporter, a balance and an analyte reactive group. The bond fragmentation pattern for the reagents is illustrated. These reagents have been determined to exhibit favorable fragmentation characteristics. Specifically, they tend to produce very little observed partially labeled analyte. Furthermore, the carbonyl balance group appears to predominately fragment by neutral loss. Moreover, because the reporter of the label comprises two basic nitrogen groups, it produces strong ion signals for both the signature ions and the labeled analytes in the mass spectrometer.

XI. Preparation of Sample Mixtures:

Once two or more samples have been differentially labeled with different isobaric labels of a set, a sample mixture can be prepared simply by mixing the samples of labeled analytes. The samples can be of any type. One or more of the samples can be control samples and one or more of the samples can be test samples. Because the unique reporter moiety of each different labeling reagent will produce a unique signature ion under MS/MS conditions, labeled analytes of each sample of the mixture can be related back to the sample from which it originated when the mixture is analyzed.

The sample mixture can be prepared by mixing differing amounts of each sample. The sample mixture can be prepared by mixing equal amounts of each of the samples. In this way there can be a direct comparison of the amount of each analyte (analyte by analyte) in each of the samples based upon the intensity of the peaks for the signature ions observed in the mass spectrometer. Whether or not equal amounts of sample are mixed together, the amount of each sample used to produce the sample mixture can be recorded. If unequal amounts of sample are used to prepare the sample mixture, appropriate ratios can be calculated from this information so that the relative and/or absolute amount (often expressed in concentration or quantity) of the analytes in the sample mixture can be determined based upon the intensity of the signature ion peaks.

For example, two or more samples containing digested peptides as the analyte, each sample being labeled with one of the isobaric labeling reagents (114, 115, 116 or 117; See FIG. 5B), were mixed to form a mixture that was analyzed in a tandem mass spectrometer. After the first MS analysis, selected ions, of a particular mass representing a mixture of fragment ions of the same analyte labeled with two or more different isobaric labels, were subjected to dissociative energy causing fragmentation of the selected ions. The selected ions, and the fragments thereof, were then re-analyzed in the mass spectrometer wherein signature ions of the isobaric labeling reagents used to label the analytes, as well as daughter ions of the analyte, were observed. Relative quantitation of analytes in the samples was determined.

XII. Sample Processing:

In certain embodiments of this invention, a sample can be processed prior to, as well as after, labeling of the analytes. The processing can facilitate the labeling of the analytes. The processing can facilitate the analysis of the sample components. The processing can simplify the handling of the samples. The processing can facilitate two or more of the foregoing.

For example, a sample can be treated with an enzyme. The enzyme can be a protease (to degrade proteins and peptides), a nuclease (to degrade nucleic acids) or some other enzyme. The enzyme can be chosen to have a very predictable degradation pattern. Two or more proteases and/or two or more nuclease enzymes may also be used together, or with other enzymes, to thereby degrade sample components.

For example, the proteolytic enzyme trypsin is a serine protease that cleaves peptide bonds between lysine or arginine and an unspecific amino acid to thereby produce peptides that comprise an amine terminus (N-terminus) and lysine or arginine carboxyl terminal amino acid (C-terminus). In this way the peptides from the cleavage of the protein are predictable and their presence and/or quantity, in a sample from a trypsin digest, can be indicative of the presence and/or quantity of the protein of their origin. Moreover, the free amine termini of a peptide can be a good nucleophile that facilitates its labeling. Other exemplary proteolytic enzymes include papain, pepsin, ArgC, LysC, V8 protease, AspN, pronase, chymotrypsin and carboxypeptidease C.

Because activity of the enzymes is predictable, the sequence of peptides that are produced from degradation of a protein of known sequence can be predicted. With this information, “theoretical” peptide information can be generated. A determination of the “theoretical” peptide fragments in computer assisted analysis of daughter fragment ions (as described below under the section entitled “Analyte Determination By Computer Assisted Database Analysis”) from mass spectrometry analysis of an actual sample can therefore be used to determine one or more peptides or proteins in one or more unknown samples.

Sample processing can involve other steps such as reducing and/or capping undesired reactive groups (e.g. thiols). For example, capping of thiol groups can be employed with labeling of peptides from digested protein so that only the amine groups, and not the thiols groups, of the peptides are labeled with the labeling reagent.

XIII. Separation of the Sample Mixture:

In some embodiments the processing of a sample or sample mixture of labeled analytes can involve separation. For example, a sample mixture comprising differentially labeled analytes from different samples can be prepared. By differentially labeled we mean that each of the labels comprises a unique property that can be identified (e.g. comprises a unique reporter moiety that produces a unique “signature ion” in MS/MS analysis). In order to analyze the sample mixture, components of the sample mixture can be separated and mass analysis performed on only a fraction of the sample mixture. In this way, the complexity of the analysis can be substantially reduced since separated analytes can be individually analyzed for mass thereby increasing the sensitivity of the analysis process. Of course the analysis can be repeated one or more time on one or more additional fractions of the sample mixture to thereby allow for the analysis of all fractions of the sample mixture.

Separation conditions under which identical analytes that are differentially labeled co-elute at a concentration, or in a quantity, that is in proportion to their abundance in the sample mixture can be used to determine the amount of each labeled analyte in each of the samples that comprise the sample mixture provided that the amount of each sample added to the sample mixture is known. Accordingly, in some embodiments, separation of the sample mixture can simplify the analysis whilst maintaining the correlation between signals determined in the mass analysis (e.g. MS/MS analysis) with the amount of the differently labeled analytes in the sample mixture.

The separation can be performed by chromatography. For example, liquid chromatography/mass spectrometry (LC/MS) can be used to perform such a sample separation and mass analysis. Moreover, any chromatographic separation process suitable to separate the analytes of interest can be used. For example, the chromatographic separation can be normal phase chromatography, reversed-phase chromatography, ion-exchange chromatography, size exclusion chromatography or affinity chromatography.

The separation can be performed electrophoretically. Non-limiting examples of electrophoretic separations techniques that can be used include, but are not limited to, 1D electrophoretic separation, 2D electrophoretic separation and/or capillary electrophoretic separation.

Isobaric labeling reagents are particularly useful when a separation step is performed because the isobaric labels of a set of labeling reagents are structurally and chemically indistinguishable (and can be indistinguishable by gross mass until fragmentation removes the reporter from the analyte). Thus, all analytes of identical composition that are labeled with different isobaric labels can chromatograph in exactly the same manner (i.e. co-elute). Because they are structurally and chemically indistinguishable, the eluent from the separation process can comprise an amount of each isobarically labeled analyte that is in proportion to the amount of that labeled analyte in the sample mixture. Furthermore, from the knowledge of how the sample mixture was prepared (portions of samples, an other optional components (e.g. calibration standards) added to prepare the sample mixture), it is possible to relate the amount of labeled analyte in the sample mixture back to the amount of that labeled analyte in the sample from which it originated.

XIV. Mass Spectrometers/Mass Spectrometry (MS):

The methods of this invention can be practiced using tandem mass spectrometers and other mass spectrometers that have the ability to select and fragment molecular ions. Tandem mass spectrometers (and to a lesser degree single-stage mass spectrometers) have the ability to select and fragment molecular ions according to their mass-to-charge (m/z) ratio, and then record the resulting fragment (daughter) ion spectra. More specifically, daughter fragment ion spectra can be generated by subjecting selected ions to dissociative energy levels (e.g. collision-induced dissociation (CID)). For example, ions corresponding to labeled peptides of a particular m/z ratio can be selected from a first mass analysis, fragmented and reanalyzed in a second mass analysis. Representative instruments that can perform such tandem mass analysis include, but are not limited to, magnetic four-sector, tandem time-of-flight, triple quadrupole, ion-trap, and hybrid quadrupole time-of-flight (Q-TOF) mass spectrometers.

These types of mass spectrometers may be used in conjunction with a variety of ionization sources, including, but not limited to, electrospray ionization (ESI) and matrix-assisted laser desorption ionization (MALDI). Ionization sources can be used to generate charged species for the first mass analysis where the analytes do not already possess a fixed charge. Additional mass spectrometry instruments and fragmentation methods include post-source decay in MALDI-MS instruments and high-energy CID using MALDI-TOF (time of flight)-TOF MS. For a recent review of tandem mass spectrometers please see: R. Aebersold and D. Goodlett, Mass Spectrometry in Proteomics. Chem. Rev. 101: 269-295 (2001). Also see U.S. Pat. No. 6,319,476, herein incorporated by reference, for a discussion of TOF-TOF mass analysis techniques.

XV. Analyte Determination by Computer Assisted Database Analysis:

In some embodiments, analytes can be determined based upon daughter-ion fragmentation patterns that are analyzed by computer-assisted comparison with the spectra of known or “theoretical” analytes. For example, the daughter fragment ion spectrum of a peptide ion fragmented under conditions of low energy CID can be considered the sum of many discrete fragmentation events. The common nomenclature differentiates daughter fragment ions according to the amide bond that breaks and the peptide fragment that retains charge following bond fission. Charge-retention on the N-terminal side of the fissile amide bond results in the formation of a b-type ion. If the charge remains on the C-terminal side of the broken amide bond, then the fragment ion is referred to as a y-type ion. In addition to b- and y-type ions, the CID mass spectrum may contain other diagnostic fragment ions (daughter fragment ions). These include ions generated by neutral loss of ammonia (−17 amu) from glutamine, lysine and arginine or the loss of water (−18 amu) from hydroxyl-containing amino acids such as serine and threonine. Certain amino acids have been observed to fragment more readily under conditions of low-energy CID than others. This is particularly apparent for peptides containing proline or aspartic acid residues, and even more so at aspartyl-proline bonds (Mak, M. et al., Rapid Commun. Mass Spectrom., 12: 837-842) (1998). Accordingly, the peptide bond of a Z-pro dimer or Z-asp dimer, wherein Z is any natural amino acid, pro is proline and asp is aspartic acid, will tend to be more labile as compared with the peptide bond between all other amino acid dimer combinations.

For peptide and protein samples therefore, low-energy CID spectra contain redundant sequence-specific information in overlapping b- and y-series ions, internal fragment ions from the same peptide, and immonium and other neutral-loss ions. Interpreting such CID spectra to assemble the amino acid sequence of the parent peptide de novo is challenging and time-consuming. The most significant advances in identifying peptide sequences have been the development of computer algorithms that correlate peptide CID spectra with peptide sequences that already exist in protein and DNA sequence databases. Such approaches are exemplified by programs such as SEQUEST (Eng, J. et al. J. Am. Soc. Mass Spectrom., 5: 976-989 (1994)) and MASCOT (Perkins, D. et al. Electrophoresis, 20: 3551-3567 (1999)).

In brief, experimental peptide CID spectra (MS/MS spectra) are matched or correlated with ‘theoretical’ daughter fragment ion spectra computationally generated from peptide sequences obtained from protein or genome sequence databases. The match or correlation is based upon the similarities between the expected mass and the observed mass of the daughter fragment ions in MS/MS mode. The potential match or correlation is scored according to how well the experimental and ‘theoretical’ fragment patterns coincide. The constraints on databases searching for a given peptide amino acid sequence are so discriminating that a single peptide CID spectrum can be adequate for identifying any given protein in a whole-genome or expressed sequence tag (EST) database. For other reviews please see: Yates, J. R. Trends, Genetics, 16: 5-8 (2000) and Yates, J. R., Electrophoresis 19: 893-900 (1998).

Accordingly, daughter fragment ion analysis of MS/MS spectra can be used not only to determine the analyte of a labeled analyte, it can also be used to determine analytes from which the determined analyte originated. For example, identification of a peptide in the MS/MS analysis can be can be used to determine the protein from which the peptide was cleaved as a consequence of an enzymatic digestion of the protein. It is envisioned that such analysis can be applied to other analytes, such as nucleic acids.

XVI. Relative and Absolute Quantitation of Analytes:

In some embodiments, the relative quantitation of differentially labeled identical analytes of a sample mixture is possible. Relative quantitation of differentially labeled identical analytes is possible by comparison of the relative amounts (e.g. area or height of the peak reported) of reporter (e.g. signature ion) that is determined in the second mass analysis for a selected, labeled analyte observed in a first mass analysis. Put differently, where each reporter can be correlated with information for a particular sample used to produce a sample mixture, the relative amount of that reporter, with respect to other reporters observed in the second mass analysis, is the relative amount of that analyte in the sample mixture. Where components combined to form the sample mixture is known, the relative amount of the analyte in each sample used to prepare the sample mixture can be back calculated based upon the relative amounts of reporter observed for the ions of the labeled analyte selected from the first mass analysis. This process can be repeated for all of the different labeled analytes observed in the first mass analysis. In this way, the relative amount (often expressed in concentration and/or quantity) of each reactive analyte, in each of the different samples used to produce the sample mixture, can be determined.

In other embodiments, absolute quantitation of analytes can be determined. For these embodiments, a known amount of one or more differentially labeled analytes (the calibration standard or calibration standards) can be added to the sample mixture. The calibration standard can be an expected analyte that is labeled with an isomeric or isobaric label of the set of labels used to label the analytes of the sample mixture provided that the reporter for the calibration standard is unique as compared with any of the samples used to form the sample mixture. Once the relative amount of reporter for the calibration standard, or standards, is determined with relation to the relative amounts of the reporter for the differentially labeled analytes of the sample mixture, it is possible to calculate the absolute amount (often expressed in concentration and/or quantity) of all of the differentially labeled analytes in the sample mixture. In this way, the absolute amount of each differentially labeled analyte (for which there is a calibration standard in the sample from which the analyte originated) can also be determined based upon the knowledge of how the sample mixture was prepared.

Notwithstanding the foregoing, corrections to the intensity of the reporters (signature ions) can be made, as appropriate, for any naturally occurring, or artificially created, isotopic abundance within the reporters. A sophisticated example of these types of corrections can be found in copending and co-owned U.S. Provisional Patent Application Ser. No. 60/524,844, entitled: “Method and Apparatus For De-Convoluting A Convoluted Spectrum”, filed on Nov. 26, 2003. The more care taken to accurately quantify the intensity of each reporter, the more accurate will be the relative and absolute quantification of the analytes in the original samples.

XVII. Proteomic Analysis:

Embodiments of this invention can be used for complex analysis because samples can be multiplexed, analyzed and reanalyzed in a rapid and repetitive manner using mass analysis techniques. For example, sample mixtures can be analyzed for the amount of individual analytes in one or more samples. The amount (often expressed in concentration and/or quantity) of those analytes can be determined for the samples from which the sample mixture was comprised. Because the sample processing and mass analyses can be performed rapidly, these methods can be repeated numerous times so that the amount of many differentially labeled analytes of the sample mixture can be determined with regard to their relative and/or absolute amounts in the sample from which the analyte originated.

One application where such a rapid multiplex analysis is useful is in the area of proteomic analysis. Proteomics can be viewed as an experimental approach to describe the information encoded in genomic sequences in terms of structure, function and regulation of biological processes. This may be achieved by systematic analysis of the total protein component expressed by a cell or tissue. Mass spectrometry, used in combination with the method, mixture, kit and/or composition embodiments of this invention is one possible tool for such global protein analysis.

For example, with a set of four isobaric labeling reagents, it is possible to obtain four time points in an experiment to determine up or down regulation of protein expression, for example, based upon response of growing cells to a particular stimulant. It is also possible to perform fewer time points but to incorporate one or two controls. It is also possible to do duplicates or triplicates in the same multiplex experiment. In all cases, up or down regulation of the protein expression, optionally with respect to the controls, can be determined in a single multiplex experiment. Moreover, because processing of the sample mixture is performed in parallel, the results are directly comparable since there is no risk that slight variations in protocol or experimental conditions may have affected the results. Accordingly, experimental analysis for which these isobaric labeling reagents can be used includes, but is not limited to, multiplex proteomic analysis, mudpit experiments, affinity pull-downs, determination of post-translational modifications (PTMs) and multiple control experiments. For example, it is possible to compare normal, diseased and/or drug treated states.

The following examples are illustrative of the disclosed compositions and methods, and are not intended to be limit the scope of the invention. Without departing from the spirit and scope of the invention, various changes and modifications of the invention will be clear to one skilled in the art and can be made to adapt the invention to various uses and conditions. Thus, other embodiments are encompassed.

### EXAMPLES

General Synthetic Notes: Unless otherwise stated, chemicals were purchased from commercial sources and used as received. Unless otherwise stated, the following chemicals were purchased from Sigma-Aldrich. Trifluoroacetic anhydride (TFAA, P/N 106232), N-Hydroxysuccinimide (NHS, P/N 13067-2), tert-Butyl bromoacetate (P/N 124230), 4-Nitrophenyl (4-NP) trifluoroacetate (P/N N22657), Pentafluorophenyl (Pfp) trifluoroacetate (P/N 377074), tert-butyldimethylsilyl (TBDMS) cyanide (407852), 1-(Trimethylsilyl)imidazole (P/N 153583), Phenyl bromoacetate (P/N 404276), Pentachlorophenol (Pcp-OH, P/N P2604), 2,2,2-Trifluoroethanol (P/N 326747), 1,1,1,3,3,3-Hexafluoro-2-propanol (HFI—OH, P/N 105228), (3-Hydroxy-1,2,3-benzotriazin-4(3H)-one (Dhbt-OH, P/N 327964), Oxalyl chloride (P/N 320420), 1-Methylpiperazine (P/N 130001), Tetrahydrofuran (THF dry, P/N 186562). Dichloromethane (DCM dry, P/N 270997), 4 M hydrochloric acid (HCl) solution in dioxane (P/N 345547), HCl (gas, P/N, 295426), 3-Nitrophenol (3-NP—OH, P/N 163031) 1,5,7-Triazabicyclo[4.4.0]dec-5-ene (TBD) bound to polystyrene crosslinked with 2% DVB, Capacity (base): ˜2.6 mmol/g (ss-TBD, Fluka, P/N 90603), H₂¹⁸O (Isotec, 95 ¹⁸O atom % (P/N 329878) or 99% ¹⁸O atom % (P/N 487090)), Br¹³CH₂COOEt (Cambridge Isotope Laboratories (CIL), P/N CLM-1010-5), Br¹³CH₂¹³COOEt (CIL, P/N CLM-1011-1), BrCH₂C¹⁸O¹⁸OH (Isotec, P/N 597031). All moisture sensitive reactions were performed under nitrogen or argon atmosphere.

Isotopically enriched starting materials were generally obtained from either Isotec (a Sigma-Aldrich company) or Cambridge Isotope Laboratories (Andover, Mass.). Generally, the most highly enriched starting materials were obtained and used in the production of the isotopically enriched piperazine derivatives. However, the state of isotopic enrichment of starting materials is a choice which the ordinary practitioner will appreciate strikes a balance between the price of the starting materials (wherein the higher the state of isotopic enrichment, the higher the price) and requirement for purity of the enriched isotopes in the final product. Accordingly, the ordinary practitioner will appreciate that the most practical method of synthesis of isotopically enriched compounds may not always proceed through the most common synthetic routes. Indeed, there may be two or more different routes to the different isotopic variants of the same compound. Thus, for some reactions and/or compounds described herein, various synthetic routes have been undertaken and are therefore discussed below. Certain advantages and caveats pertaining to these routes are also discussed.

I. Synthesis of Isotopically Labeled N-Methyl piperazines

Note: Unlabeled N-methyl piperazine (a.k.a. 1-methyl piperazine) is commercially available from a variety of sources. However, no source for any type of isotopically enriched N-methyl piperazine (as a stock item) could be found. It was determined however that suitably protected glycine and sarcosine could be condensed, cyclized and the product (a diketone) could be reduced to thereby produce N-methyl piperazine (See FIG. 1). Furthermore, it was determined that all possible permutations of ¹⁵N and ¹³C isotopically labeled glycine, as well as partially protected versions thereof (e.g. t-boc protected amino acids), were commercially available from sources such as Isotec or Cambridge Isotope Laboratory, Inc. Accordingly, this appeared to be a promising route to various N-methyl piperazine compounds comprising one or more heavy atoms. Because appropriately protected (t-boc) isotopically enriched glycines and suitably protected sarcosine can be purchased from commercial sources and because the protection of amino acids, such as glycine and sarcosine, are well-known, this discussion of the synthetic route to N-methyl piperazine begins with the suitably protected amino acids (FIG. 1).

### Example 1

### General Procedure for the Condensation of Sarcosine Ester and t-boc-Glycine (FIG. 1)

Note: Sarcosine is commercially available as either the methyl or the ethyl ester. Either can be used in the condensation reaction.

To a round bottom flask (RBF) was added 1.1 equivalent (eq.) of sarcosine ethyl ester (2) and 1 eq. of t-boc-glycine (1) (including isotopically labeled t-boc-glycines for the production of various isotopically labeled N-methyl piperazines). The solid was then dissolved with the addition of dichloromethane (DCM) (˜20 mL/g of t-boc-glycine). To this stirring solution was added 1.1 eq. of N-methyl morpholine (NMM) then 1.1 eq. of dicyclohexylcarbodiimide (DCC) in DCM. A precipitate formed within minutes. The reaction was stirred overnight. The reaction was monitored by thin layer chromatography (TLC). If t-boc-glycine was still present, additional DCC in DCM was added. When the reaction was determined to be complete, the solids were filtered off and the cake was rinsed with DCM. The product containing solution was then evaporated to dryness.

The product was purified by silica gel chromatography using a column packed in 50% ethyl acetate (EtOAc)/hexane. A small amount of the 50% EtOAc/hexane solution was used to dissolve/suspend the dried down product (not all will dissolve). This solution/slurry was loaded onto the packed column. The column was eluted 50% EtOAc/hexane to obtain the minimally retained product. Product containing fractions were evaporated to provide an oil, speckled oil, or flaky solid (materials that are higher in heavy atom isotope content appeared to exhibit more characteristics of a solid).

TLC conditions: EtOAc (developed with ninhydrin and heat)

| | |
|---|---|
| Product (3) | Rf˜0.85 |
| t-boc-glycine (1) | Rf˜0.3 (broad tailing) |
| Sarcosine-OEt (2) | Baseline |
| NMM | Faintly visible just above sarcosine |

### Example 2

### General Procedure for the Synthesis of 1-Methyl-2,5-Diketopiperazine (FIG. 1)

A solution of 1:1 trifluoroactetic acid (TFA):DCM containing 0.5% water was prepared. This solution was added to the column purified product (a) of the condensation reaction (˜10 mL/g starting material). The resulting solution was stirred for 30 minutes and then the solvents removed by rotoevaporator. Ethanol (˜10 mL/g starting material) was then added to the reaction flask and this solution was again striped to dryness. The procedure was repeated with toluene. The product was again dissolved in ethanol in the reaction flask and anhydrous potassium carbonate (4 eq) was added. The solution bubbled vigorously for a short period following the addition of the potassium carbonate. A drop of the reaction mixture was removed, diluted with water, and the pH of the solution was determined. If the pH was below 8, more potassium carbonate was added. Once the pH was confirmed to be greater than 8, the reaction was allowed to reflux overnight. The warm reaction mixture was then passed through a plug of Celite to remove the excess salts. The cake was rinsed twice with anhydrous ethanol. The filtrate was transferred to a larger flask and stripped to dryness. The product foam was redissolved in 9:1 ethyl acetate-methanol and passed through a plug of silica-gel. The silica-gel was then washed with ˜4 column volumes of 9:1 ethyl acetate-methanol. All fractions were evaporated to dryness.

Notes and alternative procedures: Deprotection of the t-boc group with the TFA/DCM/H₂O solution can be followed by TLC (ninhydrin/heat shows conversion of the spot at Rf 0.85 to a dark red-brown spot at origin). After deprotection, it is also acceptable to add methanol, concentrate and re-treat with methanol followed by a second concentration and drying in vacuo to remove excess TFA.

In some reactions, concentrated ammonium hydroxide (large excess ˜60 mL per 16 mmol of starting material) was substituted for potassium carbonate. (When concentrated ammonium hydroxide was added to the reaction at room temperature, it generated a white insoluble material and a slightly milky reaction mixture.) After the addition of concentrated ammonium hydroxide, the flask was sealed with septum to prevent loss of ammonia. Cyclization appeared to be complete after overnight (12 hrs) reaction although in some cases heating to 60° C. over several hours was sufficient. The reaction was monitored by TLC (10% MeOH/DCM visualized with 10% phosphomolybdic acid (PMA) in MeOH with heat). The product appeared as a blue spot at Rf 0.54. Since the deblocked material (red-brown spot at origin) could not be visualized with PMA, another TLC was performed as a cross-reference using ninhydrin/heat.

When cyclization was deemed complete by TLC analysis, the mixture was filtered and the flask and solids were rinsed with DCM. The filtrate was concentrated and redissolved in 10% MeOH/DCM before chromatography. The white waxy solids were partially insoluble in the 10% MeOH/DCM so the material was sonicated. Sonification successfully dissolved the mixture that was then applied to the column. The first fraction eluted was mainly the white waxy solid. The major fraction (the dione product (4)) eluted next and was followed by another minor impurity (Rf 0.3). It was observed that in cases where incomplete TFA removal resulted in formation of ammonium triflate, this impurity co-eluted with the product and the secondary material. A second column could be used to completely purify the desired product.

The melting points of the dione (4): 116,117: 138-139 for model compound: lit: 136-139 (J. Het. Chem 18, 423, 1981); 142-143 (J. Biol. Chem 61, 445, 1924).

TLC condition: 9:1 ethyl acetate-methanol (develop with phosphomolybdic acid and heat)

Product Rf˜0.2

Alternative TLC condition: 10% MeOH/DCM; develop with heat

Product (blue spot) Rf=0.54

1H-NMR data

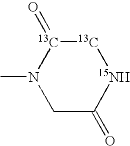

NMR (D₆ DMSO)—N1-CH₃, d 2.79, 2.80 3H; 3-CH₂ dd 3.94, 3.92, 3.59, 3.57 2H; 6-CH₂ d 3.87, 3.86 2H; 4-NH b 8.11H.

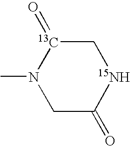

NMR (D₂O)—N1-CH₃, d 3.00, 2.99 3H; 3-CH₂ d 4.08,4.08 2H, 6-CH₂ d 4.14,4.14 2H.

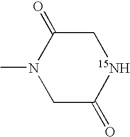

NMR (D₆ DMSO)—N-1-CH₃, s 2.80 3H; 3-CH₂ s 3.76 2H; 6-CH₂ s 3.86 2H; 4-NH d 8.03,8.26 1H.

### Example 3

### General Procedure for the Synthesis of N-Methyl Piperazine (Route A; FIG. 1)

A saturated solution of sodium sulfate was prepared. Tetrahydrofuran (THF) (4 mL per mmol starting material based upon the material used in Example 2) was added to the diketopiperazine formed using the procedure of Example 2. The reaction flask was fit with a reflux condenser and three equivalents of 1M LiAlH₄ in THF (LAH solution; it may be possible to substitute Red-Al or other reducing reagent for LAH but this has not been attempted) was added to the solution through a dropping funnel. There was vigorous hydrogen evolution at the initiation of the addition but this subsided as the addition continued. The reaction was heated to reflux for 4 hours. After the reaction was complete, the solution was cooled to room temperature and the remaining LAH was quenched with the very slow addition of saturated aqueous sodium sulfate (¼ the volume of the LAH solution added). The reaction appeared as a gray suspension.

DCM was added to this suspension (½ volume of the THF) and the gray gel-like solid was removed by filtration. The flask and filtered solids were then thoroughly washed 2× with DCM (¼ volume of the THF). The combined organic solution (DCM/THF) was then dried with Na₂SO₄ (solid-anhydrous) and filtered. (In some early experiments the N-methyl piperazine was isolated as an oil (free base and not as a TFA or HCl salt) but the product was determined to be a volatile oil and therefore not be isolated in high yield).

Di-tert-butyl-dicarbonate (3 equivalents) was added to this solution that was stirred and vented overnight. TLC was used to monitor the reaction. Once complete, the solvent was removed by rotary evaporation to yield a liquid. This liquid is slightly volatile, so low vacuum evaporation of solvent is recommended (high vacuum conditions should be avoided). The product was dissolved in DCM and loaded onto a silica-gel column packed with 8% methanol in ethyl acetate. Product was eluted with the 8% methanol in ethyl acetate solution. Product containing fractions were determined by TLC, pooled, and evaporated to a liquid. This liquid was taken directly to the deprotection reaction. Note: the t-boc deprotection was performed only as a means to isolate the crude N-methyl piperazine product but this requires subsequent deprotection.

TLC—N-methyl piperazine (develop with ninhydrin)

4:1:1 Ethanol:Water:Ammonium hydroxide

Product Rf=0.6

TLC—N¹-t-Boc-N²-methyl piperazine (develop with ninhydrin)

4:1 DCM-MeOH

Product Rf==0.5

Deprotection:

A solution of 1:1 TFA, DCM with 0.5% water was prepared. This solution was added to the material isolated from the reduction, above (˜10 mL/g starting material). The reaction was stirred for 30 minutes then the solvent was removed with a rotoevaporator. Solvent evaporation was terminated when no more solvent was observed to be collecting on the condenser. TFA was added to the product residue (˜2 mL/g starting material) to form a free flowing solution. The TFA solution was transferred to a centrifuge tube and diethyl ether was added to precipitate the product salt. The solution was mixed using a vortex. The solution was then centrifuged and the supernatant decanted to collect the precipitate. The filtrate was then washed 1 time with ether by resuspending the product, vortexing, and re-centrifugation. Product was dried under low vacuum to remove residual ether.

1H-NMR data:

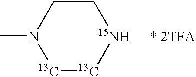

NMR (D₂O)—N1-CH₃, d 3.02,3.03 3H; methylenes, broad triplet 3.3-3.9 8H

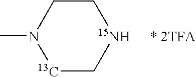

NMR (D₂O)—N1-CH₃, d 3.02,3.03 3H; methylenes, broad 3.40-3.85 8H

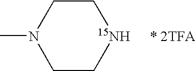

NMR (D₂O)—N1-CH₃, s 3.03 3H; methylenes, broad 3.50-3.75 8H

### Example 4

### General Procedure for the Synthesis of N-Methyl Piperazine (Route B; FIG. 1)

The product of the procedure of Example 2 was dissolved in anhydrous THF (5 mL per mmol SM) in a multi-neck RBF fitted with condenser, addition funnel and argon (Ar) inlet. To this solution was added 3 equivalent of the LAH solution slowly through a dropping funnel at RT under Ar. Vigorous hydrogen evolution was observed at the beginning. After addition, the cloudy solution was heated to reflux for 3 hours. TLC was used to determine when the reaction was complete (disappearance of starting material (SM), 10% MeOH/DCM TLC developing solvent, PMA as visualizer). After the reaction was complete, the solution was cooled to room temperature and quenched with the very slow addition of saturated aqueous sodium sulfate (¼ the volume of the LAH solution added). White gel-like solid solution was passed through a plug of Na₂SO₄ solid to remove H₂O. The filter cake was washed with THF several times (400 mL per gram SM) until TLC of the washing showed a little product. Then TFA (4 eq) was added to the THF solution (HCl in dioxane could also be added if the HCl salt was desired). The color of the solution changed to light brown from pale yellow. The solution was concentrated on a rotoevaporator under reduced pressure to yield brown oil. The light brown product was precipitated as bis-TFA salt by adding ether (42 mL per 1 gram SM) to yield of 80% N-methyl-piperazine. ¹H NMR (D₂O) was used to confirm the desired product.

II. Alkylation of N-Methyl Piperazines to Form N-Methyl Piperazine Acetic Acids

Note: FIG. 5A illustrates the pathway for the synthetic incorporation of heavy atom isotopes into four isobaric labeling reagents referred to herein as 114, 115, 116 and 117. As can be seen from FIG. 5A, certain of the heavy atom isotopes can be incorporated by the choice of the commercially available isotopically labeled glycine used in the production of the N-methyl piperazine. Certain other heavy atoms can be incorporated during the alkylation reaction based upon the choice of the commercially available bromoacetic acid. In some cases, the ¹⁸O can be incorporated through an efficient exchange using ¹⁸O labeled water. The labeling reagents are designated 114, 115, 116 and 117 based upon the mass of the fragment that forms a signature ion in the mass spectrometer (see: FIG. 5A and FIG. 5B) once the reagent has been fragmented by the application of dissociative energy.

With reference to FIG. 2A, scheme A is useful for producing the N-methyl piperazine acetic acid as a zwitterion and not as a salt (e.g. mono or bis TFA or HCl salt). With reference to FIG. 2B, scheme B is useful since it requires the use of only one equivalent of N-methyl piperazine for the production of the N-methyl piperazine acetic acid thereby foreclosing the waste of the valuable isotopically labeled starting material. With reference to FIG. 2C, scheme C is useful for alkylations involving the isotopically labeled bromoacetic acid, particularly the ¹⁸O labeled bromoacetic acid as it was expected to reduce the occurrence of ¹⁸O scrambling (or exchange with ¹⁶O from residual water).

### Example 5

### Procedure for the Synthesis of Isotopically Labeled N-Methyl Piperazine Acetic Acids (Scheme A; FIG. 2A)

To a stirring solution of 1.18 g (11.83 mmol) N-methyl piperazine in 15 mL of toluene at room temperature was added 1 g (5.91 mmol) of ethylbromoacetate,1,2-^{—}C dropwise, over a period of 15 minutes. Immediate formation of white solid was observed. The reaction mixture was then heated in an oil bath at 90° C. for 4 hr. After cooling the mixture to room temperature, the off-white solid was removed by filtration, and washed with 25 mL of toluene. The combined filtrate and washings was then concentrated in a rotoevaporator, and the residue was dried under high vacuum for 5 hours to yield 1.10 g (quantitative) of ethyl ester of N-methyl piperazine acetic acid-1,2-¹³C (9) as an off-white oil. The crude product (9) was analyzed by MS and ¹H-NMR, and was directly used for the next step without further purification. MS (ESI, m/z): 189.16 (M+1), ¹H-NMR (DMSOd₆) δ 4.2 (m, 2H), 3.4 (d,1H, J=7 Hz), 3.05 (d,1H, J=7 Hz)), 2.4-2.7 (b, 8H), 2.3 (s, 3H), 1.25 (t, 3H).

A solution of ethyl ester of N-methyl piperazine acetic acid (9) (1.1 g, mmol), prepared as described above, in water (20 mL) was refluxed for 24 hr. The reaction was monitored by MS analysis. After 24 hr, the reaction mixture was concentrated in a rotoevaporator to afford white solid product, which was triturarted with acetone (10 mL) overnight. The product was then separated by filtration and dried under high vacuum overnight at 45° C. in a vacuum oven, to yield 780 mg of N-methyl piperazine acetic acid, 1,2-^{—}C (10), as a white powdery solid. 300 mg of the product was further purified by sublimation (1 mm/Hg, 110-120° C.) to yield 270 mg of white solid. MS (ESI, m/z); 161 (M+1), ¹H-NMR (DMSOd₆) δ 3.3 (d,1H, J=7 Hz), 2.95(d, 1H, J=7 Hz), 2.55-2.75 (b,4H), 2.3-2.45 (b,4H), 2.18 (s, 3H).

Notes: This procedure utilizes unlabeled N-methyl piperazine. This procedure is useful for producing the zwitterion of N-methyl piperazine acetic acid.

The product can also be isolated as the mono or bis-HCl or mono or bis-TFA salt by treatment with the appropriate acid prior to or subsequent to its isolation as described above.

### Example 6

### Procedure for the Synthesis of Isotopically Labeled N-Methyl Piperazine Acetic Acids (Scheme B; FIG. 2B)

To a slurry of 200 mg (1.14 mmol) of N-methylpiperazine-¹⁵N2HCl (the 2TFA salt can also be used) in methanol (MeOH, 14 mL), was added 1.76 g (4.59 mmol) of ss-TBD, with a loading of 2.6 mmol/g, followed by CH₂Cl₂ (6 mL). The mixture was then sonicated for 15 minutes and was then cooled in an ice bath under an argon atmosphere. To this vigorously stirred slurry, a solution of 193 mg (1.14 mmol) of ethylbromoacetate-2-¹³C in acetonitrile (3 mL) was added dropwise using a syringe pump (maintaining a rate of 2 mL/hr). After completion of the addition, the ice bath was removed and the mixture was continued stirring at room temperature overnight (18 hr). The mixture was then filtered through a sintered funnel, and the solid was washed several times with MeOH (4×10 mL). The combined filtrate and washings were then concentrated in a rotoevaporator, and the residue was kept under high vacuum to yield 111 mg (51%) of the ethyl ester of the N-methyl piperazine acetic acid (11) as an off white solid. This crude product was directly used for the next step without further purification. MS (ESI, m/z) 189 (M+1). ¹H-NMR (DMSOd₆) δ 4.05 (q, 2H), 3.3(s,1H), 3.0 (s,1H), 2.4-2.5 (b, 4H), 2.2-2.4(b, 4H), 2.1 (s, 3H), 1.15 (t, 3H).

The product was hydrolyzed in the manner described in Scheme A, above. The following analytical data was obtained for the product.

MS (ESI, m/z) 161 (M+1). ¹H-NMR (DMSOd₆) δ 3.35(s,1H), 3.05 (s,1H), 2.65-2.8(b, 4H), 2.5-2.65(b, 4H), 2.35 (s, 3H),

Without substantial variation, above general procedure was applied to other isotopically labeled N-methyl piperazines to produce various isotopically labeled N-methyl piperazine acetic acid derivatives.

The product can also be isolated as the bis-HCl or bis-TFA salt by treatment with the appropriate acid prior to or subsequent to its isolation as described above.

### Example 7

### General Procedure for the Synthesis of Isotopically Labeled N-Methyl Piperazine Acetic Acids (Scheme C; FIG. 2C)

To a solution of bromoacetic acid (715 mg, 5 mmol) in DCM (15 mL) was added 700 mg of trityl-Cl resin (1 mmol, 1.45 mmol/g) followed by diisopropylethylamine (DIPEA) (1.79 mL, 10 mmol). This solution was mixed at room temperature for 1 hour. The resin was then filtered and washed with dichloromethane (3×4 mL) followed by a wash with a solution of dichloromethane-methanol-DIPEA (17:2:3,5 mL) and finally a wash with dichloromethane (3×4 mL).

The resin was then treated with a solution of N-methyl piperazine (N-MP) (0.57 mL, 5 mmol) in DMF (5 mL) for 30 minutes and then washed with DMF and dichloromethane (3×4 mL each). The N-MPA so generated on resin was cleaved with a 25% solution of TFA in dichloromethane (10 mL for 5 min)) and resin was washed with the same solution (2×5 mL). After evaporation of TFA, the product was precipitated and washed with ether (388 mg, 99% yield, bis TFA salt). The product was identified by NMR (matched with literature) and with ES-MS (Calculated MH⁺=159.11, found 159.14).

Without substantial variation, the above general procedure was applied to other isotopically labeled N-methyl piperazines to produce various isotopically labeled N-methyl piperazine acetic acid derivatives.

The product could also be isolated as its bis-HCl salt if HCl was used to cleave the product from the support rather than TFA. Other acids could also be used for the cleavage reaction and product would be the salt of the acid used.

III. Methods for the Incorporation of ¹⁸O into N-Methyl Piperazine Acetic Acids

Note: In the initial experiments, incorporation of ¹⁸O into the N-methyl piperazine acetic acid was attempted as illustrated in FIG. 3A using ¹⁸O labeled bromoacetic acid (custom synthesized by CIL). Caveats to this approach include the possibility that in subsequent reactions, the ¹⁸O can exchange with ¹⁶O from residual water or can otherwise exchange with ¹⁶O from other reagents during the esterification process. The more recently applied synthetic procedure is illustrated in FIG. 3B and capitalizes on the ¹⁶O [symbol] ¹⁸O exchange reaction, using H₂¹⁸O to drive the equilibrium reaction to formation of the desired heavy version of the N-methyl piperazine acetic acid. Though both schemes have been shown to work, Scheme B currently supports the production of the most highly ¹⁸O enriched products.

### Example 8

### General Procedure for the Synthesis of ¹⁸O Isotopically Labeled N-Methyl Piperazine Acetic Acids, Including Conversion to the Active Ester (Scheme A; FIG. 3A)

To a solution of TBDMS-CN (172 mg, 1.190 mmol)) in DCM (0.575 mL) was added 180 labeled bromoacetic acid (13) (170 mg, 1.189 mmol) under an argon atmosphere and the solution was heated to 80° C. for 20 minutes and then cooled to room temperature. The product (14) was isolated as an oil (254 mg, 85% yield). ¹H NMR (CDCl₃) δ 3.58 (2H, —CH₂—), 0.955 (9H, (CH₃)₃—Si), 0.30 (6H, CH₃Si).

A solution of BrCH₂CH₂C¹⁸O₂-TBDMS (14) (254 mg, 1 mmol) in DCM (2.5 mL) was added (34 μL/min) to an argon flushed flask containing N-MP (110 μL, 1 mmol), TBD resin (576 mg, 1.5 mmol, 2.6 mmol/g) and DCM at 0° C. After the addition was complete the reaction continued for 1 h at RT and then the resin was filtered and washed with DCM. Combined filtrate was concentrated by rotary evaporation to obtain 118 mg (42% yield) of an oil (15).

Note: Because of the potential for ¹⁸O [symbol] ¹⁶O exchange during the esterification, the N-methyl piperazine acetic acid prepared by this route was not converted to the active ester using the trifluoracetate procedure described in Section VI, below. Instead it was converted using oxalyl chloride and NHS as described below.

To a solution of ¹⁸O containing TBDMS ester of N-MPA (15) as obtained above (118 mg, 0.427 mmol) in DCM (5 mL) was added a solution of oxalyl chloride (0.427 mL, 0.854 mmol, 2 M solution in dichloromethane) at room temperature. The reaction allowed to continued for 1 hour when an off white slurry formed. Solvent and excess reagent were removed from the reaction mixture. A solution of NHS (50 mg, 0.427 mmol) in dry THF (1.4 mL) was added to the resulting solid followed by 5 mL of dichloromethane, 4 mL of THF and 246 mg of ss-TBD resin (0.640 mmol, 2.6 mmol/g). The mixture was sonicated and mixed for 20 minutes, after which the resin was filtered and washed with 5 mL of dry dichloromethane. To the filtrate so obtained was added 2 mL of 4.0 M solution of HCl in dioxane and the precipitate (16) was washed with dry THF (5 mL×2) and hexanes (5 mL) and dried under vacuum (10 mg, 7% yield). ES-MS (direct infusion in i-propanol) shows isotopic purity to be around 74% at this stage.

### Example 9

### General Procedure for the Synthesis of ¹⁸O Isotopically Labeled N-Methyl Piperazine Acetic Acids (Scheme B; FIG. 3B)

200 mg (1.24 mmol) of N-methyl piperazine acetic acid 1, 2-¹³C (17) was weighed out in a 5 mL plastic vial flushed with argon. The vial was then transferred into a glove box and 2.5 mL of ¹⁸O-water (>99% 180) was added. The vial was then fitted with a silicone septum, and a low stream of HCl gas was then passed through the solution using a long needle after venting the septum with an open needle. When the solution had warmed (˜2 min), the HCl passage was stopped, and the septum was replaced with a screw-cap. The vial was then heated at 80° C. in a heating block for 18 hr. An aliquot was analyzed by MS and ¹⁸O purity was calculated as 93%. The reaction mixture was then concentrated to dryness in a speedvac, and the residue was subjected to a second cycle of ¹⁸O-exchange as described above. By MS analysis the ¹⁸O purity after the second cycle was determined as 96%. The mixture was then evaporated to dryness in a speedvac, and traces of water were removed by co-evaporataion with toluene (1 mL×2). 220 mg of N-methyl piperazine acetic acid-1,2-¹³C-¹⁸O₂2HCl (18) was obtained. MS (ESI, m/z), 165 (M+1)

Note: The product was used without further purification in the production of active ester of the N-methyl piperazine acetic acid. The bis-TFA salt was also produced using the above-described procedure wherein TFA was substituted for HCl.

IV. Preparation of the Active Esters of the N-Methyl Piperazine Acetic Acids

Note: Several methods were employed for the production of active esters of N-methyl piperazine acetic acid. The procedure illustrated by Scheme A (FIG. 4A) worked well for the production of the fluoroalcohol esters of N-methyl piperazine (See: FIGS. 4C and 4D). The procedure illustrated by Scheme B (FIG. 4B) produced various active esters of N-methyl piperazine but unless the solid phase base was used (e.g. ss-TBD), the hydrochloride salt of solution phase base was difficult to remove. The procedure illustrated by Scheme C (FIGS. 4C and 4D) proved to be the most generally applicable route to the production of active esters of N-methyl piperazine.

### Example 10

### Synthesis of Active Esters of N-methyl Piperazine Acetic Acid via Imidazolide Formation (Scheme A, FIG. 4A)

To a solution of N-methyl piperazine phenyl ester (20) (100 mg, 0.426 mmol) and sodium phenoxide (1 mg, 9 μmol) in THF (5 mL) was added TMS-imidazole (69 μL, 0.468 mmol). The solution was mixed for 20 minutes to generate the imidazolide (21). CF₃CH₂OH (80 μL, 0.213 mmol) was then added to the light yellow solution so obtained. The solution was mixed for another 30 minutes when TLC indicated clean formation of product (R_{f}=0.6, 4:1 DCM-MeOH). The reaction was then diluted to 15 mL with EtOAc and the product (22) was precipitated by addition of HCl solution in dioxane (4 M, 2 mL). After washing with THF (2×15 mL) product was isolated as white solid. NMR of the solid indicated a 1:1 mixture of product and imidazole (as HCl salt). Calculated MH⁺=241.13, found=241.12.

1,1,1,3,3,3-Hexafluoro-2-propanol ester (23) was isolated using the general procedure set forth above provided however that (CF₃)₂CHOH was substituted for CF₃CH₂OH. The following analytical data was obtained for this product. (R_{f}=0.37, 9:1 DCM-MeOH). Calculated MH⁺=309.11, found=309.11.

Note: N-methyl piperazine phenyl ester was prepared by the alkylation procedures described above (See FIGS. 2A and 2B) wherein phenyl bromoacetate is substituted for ethyl bromoacetate.

### Example 11

### Synthesis of Active Esters of N-Methyl Piperazine Acetic Acid via Oxalyl Chloride (Scheme B, FIG. 4B)

To a suspension of N-methyl piperazine acetic acid (N-MPAA) (79 mg, 0.5 mmol) in DCM (25 mL) was added a solution of oxalyl chloride (4 mL, 0.8 mmol, 2.0 M solution in DCM) over 10 minute at room temperature. After another 30 minutes of reaction, solvent and excess reagent were removed under reduced pressure to give a white solid (24). A solution of NHS (57 mg, 0.5 mmol) in DCM (25 mL) was added to the solid followed by ss-TBD (390 mg, 1 mmol, 2.6 mmol/g). The resulting solution was sonicated for 5 minute when all solid dissolved. The ss-TBD resin was removed by filtration and solvent was evaporated to yield a white foam (97% yield). Product was characterized by ES-MS as before.

Synthesis of Active Esters of N-Methyl Piperazine Acetic Acid Via Trifluoroacetate Esters (Scheme C, FIGS. 4C and 4D)

Note: Conversion of the N-methyl piperazine acetic acids (N-MPAAs) to their active esters via the trifluoracetate ester is typically a two-step process. Except for the rare case where the reagent is commercially available (See: Table 1), the first step involves the preparation of a reagent for esterifying the acetic acid. The second step involves reacting the esterifying reagent with the N-methyl piperazine acetic acid to produce the active ester. Various active esters were produced and tested for the aqueous labeling of peptides. Though the NHS ester proved to be quite useful for this application, other esters may prove useful in other applications. Nevertheless, this method of producing the active esters proved to be quite robust and generally applicable across a wide variety of compounds. FIG. 4B illustrates 7 different active esters that were produced using the same generic procedure.

### Example 12

### Synthesis of N-Hydroxysuccinimide Trifluoroacetate^{10,11} and Other Trifluoroacetate Esters

Trifluoroacetic anhydride (4.9 mL, 4×8.68 mmol (2.5-4 equivalents is typically used) was added to N-hydroxysuccinimide (NHS) (1 g, 8.69 mmol) and stirred under argon for 1-2 h to produce a homogeneous reaction mixture. Excess reagent and by-product CF₃COOH were removed under reduced pressure (rotary evaporation). The product was obtained as white solid in quantitative yield. The solid was dried under high vacuum for 3-4 h and stored under argon (Ar) or nitrogen (N₂) gas.

With reference to FIG. 4D and Table 1, the trifluoroacetate ester of pentafluorophenol (Pfp) and 4-nitrophenol (4-NP) were commercially available. The remaining trifluoroacetate esters were synthesized using the above-described generic procedure provided however that the reaction time and temperature were varied. Furthermore, in some cases the products were isolated by distillation. Yields of the trifluoroacetate esters were good and in some cases near quantitative. The specific conditions used are set forth in Table 1, below.

TABLE 1
pKₐ

| | | |
|---|---|---|
| 4.68 | 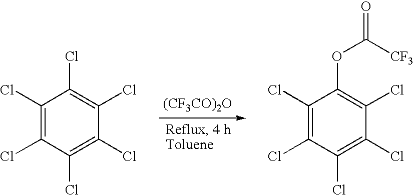 | Pcp |
| 5.50 | 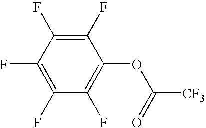 | Pfp |
| 7.23 | 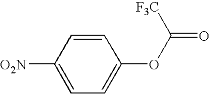 | 4-NP |
| 7.78 | 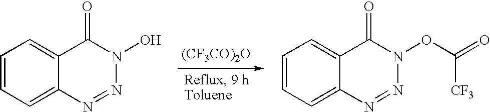 | Dhbt |
| 7.80 | 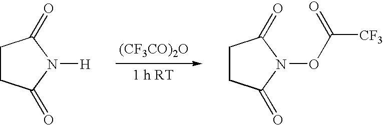 | NHS |
| 8.33 | 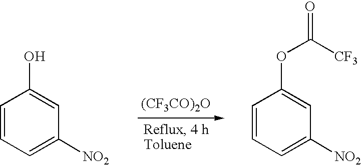 | 3-NP |
| 9.38 | 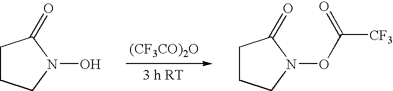 | NHP |

### Example 13

### General Method for the Preparation of Active Esters of N-Substituted Piperazine Acetic Acid from Trifluoroacetate Esters

A solution of the trifluoroacetate in THF (0.58 M, 1.2 equiv) was added to a solid sample of N-methyl piperazine acetic acid and mixed in a vortex or shaker until a homogeneous solution was obtained. The reaction of the carboxylic acid with the trifluoroacetate ester was generally complete within 30 min for all cases except N-hydroypyrrolidinone (NHP, 18 h). The progress of conversion to the active ester was monitored by ES-MS. The amount of product and any starting material (N-MPA) could be determined by direct infusion of a sample of the reaction (in ethanol) into the ES-MS. In some cases the active ester product was precipitated as dihydrochloride salt by the addition of a solution by addition of HCl solution in dioxane (4 M, 50% volume of the reaction) followed by washing with THF, ethyl acetate and hexanes. In other cases the product was isolated from the reaction as the mono TFA salt. Addition of TFA could be performed if the bis-TFA salt was desired.

| | |
|---|---|
| Dhbt ester, Calculated MH⁺ = 304.14 | Found = 304.20 |
| NHP ester, Calculated MH⁺ = 242.15 | Found = 242.20 |
| 4-NP ester, Calculated MH⁺ = 280.13 | Found = 280.20 |

¹H NMR (400 MHz, CDCl₃) d 8.20 (d, 2H, J=9.2 Hz, aromatic protons), 7.25 (d, 2H, J=9.2 Hz, aromatic protons), 3.69-3.40 (broad, 2H, ring protons), 3.57 (s, 2H, —CH₂—CO—), 3.15-2.90 (broad, 6H, ring protons), 2.78 (s, 3H, —CH₃).

| | |
|---|---|
| Pfp ester, Calculated MH⁺ = 325.10 | Found = 325.10 |
| Pcp ester, Calculated MH⁺ = 404.95 | Found = 405.90 |
| 3-NP ester, Calculated MH⁺ = 280.13 | Found = 280.20 |
| NHS ester, Calculated MH⁺ = 256.13 | Found = 256.10 |

### Example 14

### Synthesis of the NHS-Ester of N-Methyl Piperazine Acetic Acid-1,2-¹³C—¹⁸O₂, 2-HCl (the 114 Labeling Reagent)

To a slurry of N-methyl piperazine acetic acid-1,2-¹³C, ¹⁸O, 2-HCl (28) (60 mg, 0.25 mmol) in THF (1.8 mL), was added DIPEA (98 mg, 0.76 mmol) under argon. The mixture was vortexed for 5 min, and the trifluoroacetate of N-hydroxysuccinimide (160 mg, 0.76 mmol) was added. After sonicating for 10 minutes, the reaction mixture was stirred at room temperature for 4 hours, followed by a centrifugation to remove any undissolved material. The supernatant was decanted then diluted with THF (3 mL) and added slowly to a 4M solution of HCl in dioxane (1.8 mL). The precipitated HCl salt of the NHS-ester was separated by centrifugation, and washed with THF (3 mL×4), dried under high vacuum to yield 62 mg (74%) of the NHS ester (30) as an off-white solid. MS (ESI, m/z) 261 (M+1), ¹H-NMR (DMSOd₆) δ 4.05 (d,1H, J=7 Hz), 3.7 (d,1H, J=7 Hz), 3.3-3.45 (b, 2H), 2.95-3.1 (b, 2H), 2.85(s, 3H), 2.75 (m,4H).

With the exception of using a different isotopically enriched N-methyl piperazine acetic acid, the above describe procedure was followed for the production of the 115 labeling reagent (31). The analytical data for the product (31) is as follows.

MS (ESI, m/z) 261 (M+1). ¹H-NMR (DMSOd₆) δ 4.05(s,1H), 3.7 (s,1H), 3.3-3.4 (b, 2H), 3.1-2.95(b, 4H), 2.85 (s, 3H), 2.75-2.80 (b, 1H), 2.7 (m, 4H).

Notes: The trifluroacetate ester reagent can be reacted with the zwitterion of N-methyl piperazine acetic acid and well as with a mono salt or bis salt (e.g. mono-HCl salt, mono-TFA salt, bis-HCl salt or bis-TFA salt) of the N-methyl piperazine acetic acid. When the bis-HCl salt was used a base such as diisoproplyethylamine (DIPEA) was added to neutralize the acid. The transesterification reaction did however apparently proceed with the bis-TFA salt of N-methyl piperazine acetic acid without the addition of base.

V. Labeling and Analysis Using a Set of Isobaric Reagents

### Example 15

### General Procedure for the Labeling of Peptide Mixtures (from Protein Digestion) with the NHS-Ester of N-Substituted Piperazine Acetic Acids (NMPAA-NHS)

Buffer and Reagent Compositions

| Reagent | Composition |
|---|---|
| Dissolution Buffer | 0.5 M triethylammonium bicarbonate, pH 8.5 |
| Denaturing Reagent | 2% sodium dodecyl sulfate (SDS) in water |
| Reducing Reagent | 50 mM neutralized tris[2-carboxyethyl] phosphine |
| | (TCEP) in water |
| Cysteine Blocking | 200 mM methyl methanethiosulfonate (MMTS) in |
| Reagent | 2-propanol |
| Trypsin | 25 μg TPCK-treated trypsin, 222 μg calcium |
| | chloride |
| NMPAA-NHS | Approximately 1 mg active reagent/tube |

Reducing the Proteins and Blocking Cysteine

Note: If necessary add up to 100 μL Dissolution Buffer to dissolve the sample in steps 1 and 2 below. Dry down the excess buffer before labeling (see step 6 in “Digesting with Trypsin.”)
1. Add 20 μL of the Dissolution Buffer to a tube containing 100 μg of the Control sample. (If working with a concentrated sample solution, add Denaturing Buffer to bring the volume up to 20 μL.)
2. Add 20 μL of the Dissolution Buffer to a tube containing 100 μg of the Test sample. (If working with a concentrated sample solution, add Denaturing Buffer to bring the volume up to 20 μL.)
3. Add 1 μL of the Denaturing Reagent to both the Control and Test sample tubes.
4. Add 2 μL of the Reducing Reagent to both the Control and Test sample tubes.
5. Vortex to mix, then spin.
6. Incubate Control and Test tubes at 60° C. for 1 hour.
7. Vortex to mix, then spin.
8. Add 1 μL Cysteine Blocking Reagent to each tube.
9. Vortex to mix, then spin.
10. Incubate at room temperature for 10 minutes.
11. Vortex to mix, then spin the Control and Test tubes.
   Digesting with Trypsin
1. Reconstitute a vial of trypsin with calcium chloride with 25 μL of Milli-Q® water or equivalent.
2. Vortex to mix, then spin.
3. Add 10 μL of the trypsin solution to each of the protein solutions.
4. Vortex to mix, then spin.
5. Incubate 12 to 16 hours at 37° C.
6. Vortex to mix, then spin.

Note: If you increased the denaturing buffer in steps 1 and 2 of “Reducing the Proteins and Blocking Cysteine,” dry the samples in a centrifugal vacuum concentrator. To the dried sample, add 30 μL of Denaturing Buffer, vortex to mix, then spin.

Labeling with the NHS Esters of NMPAA (NMPAA-NHS)

Note: Bring a vial of NMPAA-NHS Reagent 114 and a vial of NMPAA-NHS Reagent 117 to room temperature (the vial should contain approximately 1 mg of active reagent).
1. Add 70 μL of absolute ethanol to each reagent vial.
2. Vortex for 1 minute to dissolve, then spin.
3. Transfer the solution of NMPAA-NHS Reagent 114 to the Control sample solution.
4. Transfer the solution of NMPAA-NHS Reagent 117 to the Test sample solution.
5. Vortex each vial to mix, then spin.
6. Incubate for 1 hour at room temperature.
7. Vortex each vial to mix, then spin.
8. Combine the contents of the NMPAA-NHS Reagent 114 vial and the NMPAA-NHS Reagent 117 vial in one tube.

Note: If a greater number of samples or controls is desired, the NMPAA-NHS Reagent 115 and NMPAA-NHS Reagent 116 can also be used.

Final Concentration of Components in Labeling Solution (104 μL Total Volume):

96 mM triethylammonium bicarbonate

0.019% SDS

0.96 mM TCEP

1.9 mM MMTS

10 μg trypsin

89 μg calcium chloride

1 mg NMPAA-NHS Reagent

1 μL 2-propanol

70 μL ethanol (67%)

100 μg sample

### Example 16

### Yeast Protein Extraction and Labeling

Yeast strains were provided by Allan Jacobson at U. Mass. Medical School, Worcester Mass. Cells were harvested, frozen and mechanically lysed by grinding over dry ice. Crude cell lysate was prepared by suspending frozen cellular material in protein buffer (0.25 M TEAmB, 0.1% SDS, 6M Guanidine), vortexing and pelleting insoluble debris by centrifugation at 10,000 g for 10 minutes. Protein in the supernatant was reduced (2 mM TCEP), alkylated (5 mM iodoacetamide) and acetone precipitated. After resuspension in digestion buffer (100 mM TEAmB, 0.05% SDS), the protein content was quantitated (BCA assay, BioRad, Hercules Calif.) and 500 mg amounts from each yeast strain in an equal volume was trypsin digested for 18 hours and freeze dried in 50 μg aliquots. For each strain, 150 μg of digest was resuspended in 100 μL of labeling buffer (0.25 M TEAmB, 75% Ethanol), after which 1 mg of isotopically enriched NHS ester of N-methylpiperazine acetic acid (the labeling reagent) was added and allowed to react at room temperature for 30 minutes. Each sample was labeled with a different isobaric labeling reagent of the set of isobaric labeling reagents. Residual labeling reagent was quenched by the addition of 300 μl of water and the 3 labeled samples were mixed and freeze dried.

The examples set forth above are for illustrative purposes only and should not be viewed as a limitation on the scope of the invention.

### REFERENCES

1 (a) Finkelstein, J. A.; Kruse, L. I.; Leonard, T. B. Dopamine Beta-hydroxylase Inhibitors EP 0261804-A1, 1987 (b) Rautio, J.; Nevalainen, T.; Taipale, H.; Vepsalainen, J.; Gynther, J.; Laine, K.; Jarvinen, T. Synthesis and in vitro Evaluation of Novel Morpholinyl- and m-Methylpiperazinylacyloxyalkyl Prodrugs of 2-(6-Methoxy-2-naphthyl)propionic acid (Naproxen) for Topical Drug Delivery. J. Med. Chem. 2000, 43, 1489-1494.
2 Nudelman, A.; McCaully, R. J.; Bell, S. C. Preparation of 1,4-Benzodiazepines. American Home Products Corp. U.S. Pat. No. 3,860,581. Jan. 14, 1975.
3 Wissner, A.; Grudzinskas, C. V. Reaction of tert-Butyldimethylsilyl Esters with Oxalyl Chloride-Dimethylformamide: Preparation of Carboxalic Acid Chlorides under Neutral Conditions. J. Org. Chem. 1978, 43, 3972-3974.
4 Heyes, M. P.; Markey, S. P. (18O)-Quinolinic acid: its esterification without back exchange for use as internal standard in the quantification of brain and CSF quinolinic acid. Biomedical & Environmental Mass Spectrometry, 1988, 15, 291-293.
5 Biswas, A.; Miller, M. J. Rearrangement of N-(p-Toluenesulfonyloxy)-2-Pyrrolidinone. Heterocycles, 1987, 26, 2849-2851.
6 (a) Stacey, M.; Bourne, E. J.; Tatlow, J. C.; Tedder, J. M. A General Method of Esterification using Trifluoroacetic anhydride. Nature 1949, 164, 705. (b) Sakakibara, S. Nukai, N. A New Reagent for the p-Nitrophenylation of Amino Acids. Bull. Chem. Soc. Jpn. 1964, 37, 1231.
7 Krusic, P. J.; Chen, K. S.; Meakin, P.; Kochi, J. K. Electron Spin Resonance Studies of Fluoroalkyl Radicals in Solution. III. Photolysis of Perfluoroketones and Adduct Formation. J. Phys. Chem. 1974, 78, 2036-2047.
8 Bates, G. S.; Diakur, J.; Masamune, S. Selective and Direct Activation of O-esters. Conversion of Phenyl and 2,2,2-Trifluoroethyl Esters into Acyl Imidazolides. Tetrahedron Letters, 1976, 49, 4423-4426.
9 Bishop, B. F. New Antiparasitic Agents Related to the Milbemycins and Avermectins. Pfizer Ltd. WO9415944, 21 Jul. 1994.
10 Rao, T. Sudhakar; Nampalli, Satyam; Sekher, Padmanabhan; Kumar, Shiv. TFA-NHS as Bifunctional Protecting Agent: Simultaneous Protection and Activation of Amino Carboxylic Acids. Tetrahedron Letters 2002, 43, 7793-7795.
11 Sakakibara, Shumpei; Inukai, Noriyoshi. Trifluoroacetate Method of Peptide Synthesis. I. The Synthesis and Use of Trifluoroacetate Reagents. Bulletin of the Chemical Society of Japan, 1965, 38, 1979-1984.

1. A mixture comprising the same analyte labeled with two or more different isobaric labels, wherein at least two of the labeled analytes are compounds of the formula selected from the group consisting of: 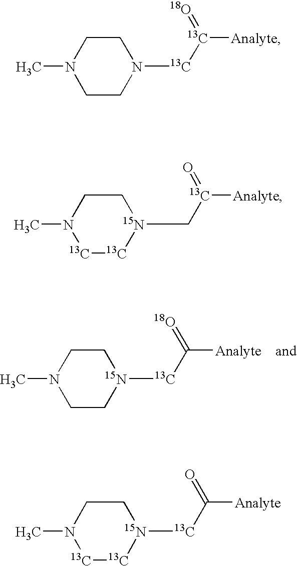
or a salt thereof.

2. The mixture of claim 1, wherein the analyte is a peptide.

3. The mixture of claim 1, wherein the analyte is a protein.

4. The mixture of claim 1, wherein the analyte is a nucleic acid.

5. The mixture of claim 1, wherein the analyte is a carbohydrate, lipid or steroid.

6. The mixture of claim 1, wherein the analyte is a small molecule with a molecular weight of less than 1500 daltons.

7. A mixture of fragment ions of the same analyte labeled with two or more different isobaric labels selected for fragmentation and further analysis in a tandem mass spectrometer, wherein at least two of the labeled analytes are compounds of a formula selected from the group consisting of: 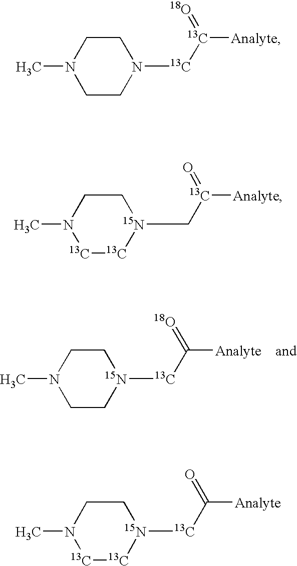
wherein all ion fragments are either positively or negatively charged.

8. The mixture of claim 7, wherein the analyte is a peptide.

9. The mixture of claim 7, wherein the analyte is a protein.

10. The mixture of claim 7, wherein the analyte is a nucleic acid.

11. The mixture of claim 7, wherein the analyte is a carbohydrate, lipid or steroid.

12. The mixture of claim 7, wherein the analyte is a small molecule with a molecular weight of less than 1500 daltons.

13. The mixture of claim 7, wherein the fragmentation and further analysis produces at least two signature ions of the molecular formula selected from the group consisting of: ¹³CC₅H₁₃N₂³⁰, ¹³CC₅H₁₃¹⁵NN⁺, ¹³C₂C₄H₁₃¹⁵NN⁺ and ¹³C₃C₃H₁₃¹⁵NN⁺.
